(19) United States
(12) Patent Application Publication
(10) Pub. No.: US 2021/0017248 A1
(43) Pub. Date: Jan. 21, 2021

(54) CELLULAR SIGNALING DOMAIN ENGINEERING IN CHIMERIC ANTIGEN RECEPTOR-MODIFIED REGULATORY T CELLS

(71) Applicant: THE REGENTS OF THE UNIVERSITY OF CALIFORNIA, San Francisco, CA (US)

(72) Inventors: Jeffrey A. Bluestone, San Francisco, CA (US); Qizhi Tang, San Francisco, CA (US); Leonardo M.R. Ferreira, San Francisco, CA (US)

(21) Appl. No.: 16/981,168

(22) PCT Filed: Mar. 15, 2019
(86) PCT No.: PCT/US2019/022546

§ 371 Date: Sep. 15, 2020

Related U.S. Application Data
(60) Provisional application No. 62/644,290, filed on Mar. 16, 2018.

(51) Int. Cl. C07K 14/725; C07K 14/705; C07K 16/28; C12N 5/0783; A61K 35/17; A61P 37/06

(52) U.S. Cl. CPC C07K 14/7051; C07K 14/70578; C07K 16/2803; C12N 5/0636; C07K 14/70521; C12N 2510/00; A61P 37/06; C07K 2319/03; C07K 2319/02; C07K 2319/33; C07K 2317/622; A61K 35/17

(57) ABSTRACT

Chimeric antigen receptor (CAR)-expressing T regulatory cells (Tregs) include intracellular co-stimulatory or inhibitory domains based on the biology, functions and activities of Tregs. The co-stimulatory or inhibitory domains modulate the Treg response, thereby, activating or suppressing an effector T cell (Teff) immune response to specific antigens.

### FIELD OF THE INVENTION

Embodiments of the invention are directed to chimeric antigen receptors (CAR) and their signaling components for the regulation of an immune response. In particular, signaling domains engineered in chimeric antigen receptor-modified regulatory T cells and theirs use in treating autoimmune disorders, inflammatory diseases, and transplant rejection.

### BACKGROUND

Manipulating human regulatory T cells (Tregs) offers an opportunity to induce tolerance in a clinical setting. However, low numbers of antigen-specific Tregs and Treg instability upon prolonged expansion have hampered the implementation of Treg-based therapies. Chimeric antigen receptor (CAR) technology has expedited the generation of tumor antigen-specific effector T (Teff) cells. CARs are recombinant receptors comprising an antigen-binding domain and an intracellular signaling domain.

### SUMMARY

Embodiments of the invention are directed, in part, to the generation of CAR-Tregs for effective antigen-specific immune tolerance induction in the contexts of, but not limited to, organ-specific autoimmune and autoinflammatory disorders (such as type 1 diabetes, RA, vitiligo), graft-versus-host disease, and immunosuppression-free organ and tissue transplantation. In some embodiments, the intracellular signaling domain is or includes a primary signaling domain, a signaling domain that is capable of inducing a primary activation signal in a T cell. The intracellular signaling domain may include an intracellular signaling domain of a CD3 chain, optionally a CD3-zeta (CD3ζ) chain, or a signaling portion thereof. In some embodiments, the intracellular signaling domain further includes a second signaling domain. In some embodiments, the second signaling domain is a costimulatory signaling domain that may include an intracellular signaling domain of a CD28, or a signaling portion thereof.

In one aspect, a chimeric antigen receptor (CAR) is provided comprising an antigen specific binding domain, a spacer domain, a transmembrane domain, and an intracellular signaling region; the signaling region comprising a primary signaling domain, optionally derived from a CD3 chain domain including CD3 γ, δ, ε, ζ and chains, and a second signaling domain which is a costimulatory alone or in combination with other costimulatory or inhibitory signaling domain of a protein selected from the group consisting of: CD28, ICOS, CTLA4, 41BB, CD27, CD30, CD132, OX-40, TACI, GITR, HVEM, TIM3, PD1, LAG3, TIGIT, and derivatives, mutants, variants, fragments and combinations thereof.

In a second aspect, an isolated T cell is provided that is modified to express: a chimeric antigen receptor (CAR) comprising an antigen specific binding domain, a spacer domain, a transmembrane domain, and an intracellular signaling region, the signaling region comprising a primary signaling domain, optionally derived from a CD3 chain domain, and a second signaling domain which is a costimulatory or inhibitory signaling domain of a protein selected from the group consisting of: CD28, ICOS, CTLA4, 41BB, CD27, CD30, CD132, OX-40, TACI, GITR, HVEM, TIM3, PD1, LAG3, TIGIT, and derivatives, mutants, variants, fragments and combinations thereof.

In a third aspect, a chimeric antigen receptor is provided, comprising an antigen binding domain and at least one signaling domain, wherein the signaling domain is a costimulatory or inhibitory signaling domain of a protein selected from CD28, 41BB, TACI, HVEM, GITR, OX40, CD27, CD30, and CD3 γ, δ, ε, ζ chain, and derivatives, mutants, variants, fragments and combinations thereof.

In a fourth aspect, a chimeric antigen receptor is provided, comprising an antigen binding domain and at least one signaling domain, wherein the signaling domain is a costimulatory or inhibitory signaling domain of a protein selected from CD28, CTLA4, PD1, TIM3, LAG3, TIGIT, and CD3 γ, δ, ε, ζ chain, and derivatives, mutants, variants, fragments and combinations thereof.

In a fifth aspect, a chimeric antigen receptor comprising an antigen binding domain and at least one signaling domain, wherein the signaling domain is a costimulatory or inhibitory signaling domain of a protein selected from CD28, CD132, and CD3 γ, δ, ε, ζ chain, and derivatives, mutants, variants, fragments and combinations thereof.

In a sixth aspect, a chimeric antigen receptor is provided, comprising an antigen binding domain and at least one signaling domain, wherein the signaling domain is a costimulatory or inhibitory signaling domain of a protein selected from ICOS, 41BB, and a CD3 γ, δ, ε, ζ chain, and derivatives, mutants, variants, fragments and combinations thereof.

In a seventh aspect, a chimeric antigen receptor is provided, comprising an antigen binding domain and at least one signaling domain, wherein the signaling domain is a costimulatory or inhibitory signaling domain of a protein selected from CTLA4, 41BB, and CD3 γ, δ, ε, ζ chain, and derivatives, mutants, variants, fragments and combinations thereof.

In an eighth aspect, an expression vector encoding any one of the chimeric antigen receptors embodied herein, is provided.

In a ninth aspect, a host cell comprising an expression vector encoding any one of the chimeric antigen receptors embodied herein, is provided.

In a tenth aspect, a method is provided for treating a subject suffering from an autoimmune and/or inflammatory disease or disorder, comprising: isolating and separating CD4⁺ T regulatory (Treg) cells from a biological sample, wherein the Treg cells are CD4⁺ CD25⁺ CD127⁻; contacting the Treg cells with an expression vector encoding a chimeric antigen receptor (CAR) which specifically binds to an antigen associated with an autoimmune response and/or suppresses an effector T (Teff) cells or inflammatory immune response; stimulating the Treg cells with a specific antigen to obtain a therapeutically effective number of antigen-specific Treg cells; and, reinfusing the Treg cells into the subject, thereby treating the subject.

In an eleventh aspect, a method is provided for treating a subject suffering from graft versus host disease, and/or is undergoing an organ transplantation, comprising: isolating and separating CD4⁺ T regulatory (Treg) cells from a biological sample, wherein the Treg cells are CD4⁺CD25⁺CD127⁻; contacting the Treg cells with an expression vector encoding a chimeric antigen receptor (CAR) which specifically binds to transplant antigens and/or suppresses an effector T (Teff) cell immune response; stimulating the Treg cells with a specific antigen to obtain a therapeutically effective number of antigen-specific Treg cells; and, reinfusing the Treg cells into the subject, thereby treating the subject.

In a twelfth aspect, a pharmaceutical composition is provided, having an isolated T cell is provided that is modified to express a chimeric antigen receptor (CAR) of the invention, together with a pharmaceutically acceptable carrier.

Other aspects are described infra.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the term “affinity” is meant a measure of binding strength. Without being bound to any one theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, including use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

As used herein, the term “agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the invention at any stage during clinical trials, during pre-trial testing, or following FDA-approval.

“Ameliorate” is meant decrease, suppress, attenuate, diminish, arrest, or stabilize the development or progression of a disease.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

As used in this specification and the appended claims, the term “or” is generally employed in its sense including “and/or” unless the content clearly dictates otherwise.

The term “chimeric antigen receptor” or “CAR” as used herein refers to recombinant receptors that generally contain an extracellular antigen-binding domain and an intracellular signaling domain. In certain embodiments, the CAR also comprises a transmembrane domain. In certain embodiments the CAR's extracellular antigen-binding domain is composed of a single chain variable fragment (scFv) derived from a fusion protein of the variable regions of the heavy and light chains of an antibody. Alternatively, scFvs may be used that are derived from Fab fragments (instead of from an antibody, e.g., obtained from Fab libraries). In various embodiments, the scFv is fused to the transmembrane domain and then to the intracellular signaling domain. “First-generation” CARs include those that solely provide CD3-chain induced signal upon antigen binding. “Second-generation” CARs include those that provide both CD3-chain induced signal upon antigen binding and co-stimulation, such as one including an intracellular signaling domain from a costimulatory receptor (e.g., CD28 or 41BB). “Third-generation” CARs include those that include multiple co-stimulatory domains of different costimulatory receptors. A fourth generation of CAR T cells include CAR T cells redirected for cytokine killing (TRUCK) where the vector containing the CAR construct possesses a cytokine cassette. When the CAR T cell is activated, the CAR T cell deposits a pro-inflammatory cytokine into the tumor lesion. A CAR-T cell is a T cell that expresses a chimeric antigen receptor. The terms “artificial T-cell receptor,” “chimeric T-cell receptor,” and “chimeric immunoreceptor” may each be used interchangeably herein with the term “chimeric antigen receptor.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “disease” is a state of health of an animal wherein the animal cannot maintain homeostasis, and wherein if the disease is not ameliorated then the animal's health continues to deteriorate. Examples of diseases include autoimmune diseases such as, rheumatoid arthritis (RA), inflammatory bowel disease (IBD), Crohn's disease (CD), ankylosing spondylitis (AS), and the like.

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208 (2005).

The term “hinge” or “hinge region” refers to a flexible polypeptide connector region providing structural flexibility and spacing to flanking polypeptide regions. The hinge can consist of natural or synthetic polypeptides.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells).

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “linker”, also referred to as a “spacer” or “spacer domain” as used herein, refers to an amino acid or sequence of amino acids that that is optionally located between two amino acid sequences in a fusion protein of the invention.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

As used herein, the term “single-chain variable fragment” or “scFv” is a fusion protein of the variable regions of the heavy (VH) and light chains (VL) of an immunoglobulin covalently linked to form a VH::VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. In some embodiments, the linker includes glycine for flexibility, and serine or threonine for solubility. scFv proteins retain the specificity of the original immunoglobulin. Single chain Fv antibodies can be expressed as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) (2008) 27(6):455-51; Peter et al., J Cachexia Sarcopenia Muscle (2012) Aug. 12; Shieh et al., J Imunol (2009) 183(4):2277-85; Giomarelli et al., Thromb Haemost (2007) 97(6):955-63; Fife et al., J Clin Invst (2006) 116(8):2252-61; Brocks et al., Immunotechnology (1997) 3(3):173-84; Moosmayer et al., Ther Immunol (1995) 2(1):31-40). Agonistic scFvs having stimulatory activity have been described (see, e.g., Peter et al., J Biol Chem (2003) 25278(38):36740-7; Xie et al., Nat Biotech (1997) 15(8):768-71; Ledbetter et al., Crit Rev Immunol (1997) 17(5-6):427-55; Ho et al., BioChim Biophys Acta (2003) 1638(3):257-66).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In preferred embodiments, the genes, nucleic acid sequences, amino acid sequences, peptides, polypeptides and proteins are human. The term “gene” is also intended to include variants.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

### BRIEF DESCRIPTION OF THE DRAWINGS

FIG. 1 is a schematic representation showing constructs including multiple signaling domains.

FIG. 2 is a schematic representation showing a schematic of various CAR constructs including multiple signaling domains.

FIG. 3 is a schematic of a timeline of in vitro stimulation of CAR Tregs. Tregs and Teff cells were FACS sorted from peripheral blood mononuclear cells (PBMCs) and stimulated with anti-CD3/CD28 beads in the presence of IL-2. Nine days later, expanded Tregs were stimulated a second time with anti-CD3/CD28 beads. On Day 11, cells were transduced with a lentiviral vector to stably express CD19 CAR. On Day 18, transduction efficiency and activation status were assessed by flow cytometry, and CD19 CAR Tregs were co-incubated with CD19-expressing WIC-negative CD80/CD86-negative stimulatory (K562) cells in the presence of IL-2, to sustain Treg survival, and CTLA4-Ig (Belatacept), to block interactions via endogenous CD28. Control stimulation cultures are identical expect the stimulatory K562 cells do not express CD19.

FIG. 4 shows that CAR-mediated CD3 signaling affects expansion of CAR Tregs in vitro. CD19 CAR-expressing Tregs including different CD3 chains downstream of CD28 were co-incubated with either K562 or CD19-K562 cells for two weeks. Left: CD71 (activation marker) MFI (×1000) measured two days after co-incubation with CD19-K562 K562 cells. Center: Representative experiment showing CD71 levels across constructs. Light color indicates incubation with parental K562 cells, while darker color indicates CD19-K562 cells. Right: CD19 CAR-expressing Treg numbers at the end of the co-incubation experiment (Day 14). UT, untransduced.

FIG. 5 is a schematic for canonical motifs found in the CD28 cytoplasmic tail. The three highlighted sites are mutated singly or in combination to construct CARs tested in experiments shown in FIGS. 6 and 7.

FIG. 6 shows that intact CAR-mediated CD28 signaling is required for optimal activation and expansion of CAR Tregs in vitro. CD19 CAR-expressing Tregs harboring different mutation in the CD28 CAR endodomain were co-incubated with either K562 or CD19-K562 cells for two weeks. Activation markers measured two days after co-incubation with either CD19-K562 or parental K562 cells. Left: CD71 (activation marker) measured two days after co-incubation with CD19-K562 cells. Center: Representative experiment showing CD71 levels across constructs. Light color (−) indicates incubation with parental K562 cells, while darker color (+) indicates CD19-K562 cells. Right: CD19 CAR-expressing Treg numbers at the end of the co-incubation experiment (Day 14). UT, untransduced.

FIG. 7A are graphs showing the fold change in CD71 and CD25 (activation markers) MFI in CD19 CAR-expressing Teff two days after co-incubation with CD19-K562 cells. FIG. 7B are graphs that illustrating that mutating the canonical motifs of the CD28 endodomain of the CD19 CAR lead to the same pattern of early activation levels (Day 2 post co-incubation with CD19-expressing target cells) in Tregs and Teff cells, as assessed by CD71 surface expression. Likewise, changing the CD3 chain of the CAR from zeta to delta or epsilon also led to the same quantitative difference in activation levels. Of note, the positive change in magnitude of early activation from zeta only (first generation) to CD28-zeta (second generation) signaling is much greater for Tregs than for Teff cells, indicating that CAR Tregs are more dependent on CD28 costimulation via the CAR than CAR Teff cells. In addition, ablating all three canonical motifs in the CD28 endodomain did not completely ablate the contribution of CD28 to activation in CAR Tregs, suggesting the existence of yet to be identified motifs. FIG. 7C shows a summary table where each column is a different CAR signaling architecture (CD28 mutant series and CD3 chain series) and each row is a different early activation marker (assessed by flow cytometry). The x axis represents time, in days of co-incubation with CD19-expressing target cells, and the y axis represents fold change in expression over untransduced T cells. Tregs are in black and Teff cells in grey.

FIG. 8A shows CD71 expression of CD19 CAR-expressing Tregs and CD19 CAR-expressing Teff cells after two days of incubation with CD19-K562 (dark color) or parental K562 cells (light color). FIG. 8B shows levels of early activation markers in CAR Tregs after two days of co-incubation with CD19-expressing target cells. FIG. 8C shows a summary table where each column is a different CAR signaling architecture and each row is a different early activation marker (assessed by flow cytometry). The x axis represents time, in days of co-incubation with CD19-expressing target cells, and the y axis represents fold change in expression over untransduced T cells. Tregs are in black and Teff cells in grey. Of note, the positive change in magnitude of early activation from zeta only (first generation) to CD28-zeta (second generation) signaling is much greater for Tregs than for Teff cells, indicating that costimulation via the CAR has a stronger impact on Tregs than on Teff cells.

FIG. 9 shows IL-10 secretion by CD19 28z CAR-expressing Tregs, but not by CD19 41BBz CAR-expressing Tregs, post stimulation with CD19-K562 cells.

FIG. 10 is a graph showing the CD19 CAR-expressing Treg and CD19 CAR-expressing Teff cell number after 14 days of co-culture with irradiated CD19-K562 cells.

FIG. 11 shows expression levels of (intracellular) FOXP3, (intracellular) CTLA4, and CD38 on CD19 CAR-expressing Tregs after 14 days of co-culture with irradiated CD19-K562 cells.

FIG. 12 shows that CD71 expression of CD19 CAR-expressing Tregs and CD19 CAR-expressing Teff cells after two days of incubation with CD19-K562 (dark color) or parental K562 cells (light color).

FIG. 13 is a schematic representation showing that internalization of CTLA4 is mediated by its YVKM intracellular motif.

FIG. 14 shows the CD71 expression of CD19 CAR-expressing Tregs and CD19 CAR-expressing Teff cells after two days of incubation with CD19-K562 (dark color) or parental K562 cells (light color).

FIG. 15A shows the CD71 levels on “Day 0” before co-incubation with irradiated CD19-K562 cells. Each colored dot is an independent experiment. FIG. 15B shows a representative CD19 CAR-expressing Treg experiment displaying expression of CD25, CD71 and ICOS before co-incubation with irradiated CD19-K562. The arrow indicates the CD71 histogram for CD19 28-30z CAR-expressing Tregs.

FIG. 16 is a graph showing that CD71 levels in co-cultures of CD19 CAR-expressing Tregs with CD19-K562 over time. CD19 28-30z CAR-expressing Tregs reached maximum CD71 expression similar to CD19 28z CAR-expressing Tregs, but with a delay.

FIG. 17 shows that CD71 expression of CD19 CAR-expressing Tregs and CD19 CAR-expressing Teff cells after fourteen days of co-incubation with CD19-K562 cells. CD19 28-30z CAR sustained elevated CD71 levels in Teff by Day 14.

FIG. 18 shows the CD71 expression of CD19 CAR-expressing Tregs and CD19 CAR-expressing Teff cells after two days of incubation with CD19-K562 (dark color) or parental K562 cells (light color).

FIG. 19 shows the CD71 expression of CD19 CAR-expressing Tregs and CD19 CAR-expressing Teff cells after two days of incubation with CD19-K562 (dark color) or parental K562 cells (light color).

FIG. 20 shows the CD19 CAR-expressing Treg and CD19 CAR-expressing Teff cell number on Day 14 of co-incubation with CD19-K562 cells. Note that CD19 28-TIGITz CAR appeared to promote expansion of Tregs and not Teff, whereas CD19 41BBz CAR appeared to promote expansion of Teff and not Tregs. CD19 28-PD1z CAR did not appear to promote proliferation in either Tregs or Teff.

FIG. 21 is a series of graphs that show that including CTLA4 signaling in the CAR provides a different outcome depending on whether it is combined with CD28 or with 41BB signaling.

FIG. 22A shows that Tregs remain highly suppressive in vitro regardless of CAR expression, as measured by inhibition of Teff cell proliferation in response to anti-CD3/CD28 dynabeads. Proliferation was measured by tritiated thymidine incorporation after 3.5 days of co-incubation. FIG. 22B demonstrates that CD19 CAR Tregs, but not mock-transduced Tregs, inhibit CD19 CAR Teff cell proliferation in vitro upon CAR-mediated activation by anti-CD19 CAR idiotype beads. Proliferation was measured by tritiated thymidine incorporation after 3.5 days of co-incubation. FIG. 22C shows that CD19 CAR-expressing Tregs efficiently inhibit CAR T cell-mediated graft rejection in vivo. 8-12 week old NSG mice were injected subcutaneously with three million CD19-K562 cells. Ten days later, CD19 CAR-expressing T cells were injected intravenously (retro-orbital route) either with or without CD19 CAR-expressing Tregs. CD19-28z CAR-expressing T cells alone led to tumor rejection (as assessed by tumor volume) within 2 weeks (black line), unless co-administered with CD19-28z CAR-expressing Tregs (red line). CD19 CAR-expressing Tregs harboring different CD19 CAR constructs illustrated in FIG. 2 are expected to prevent rejection to varying degrees.

FIG. 23A represents the levels of 32 different cytokines in the supernatant of CD19 CAR Tregs and CD19 CAR Teff cells transduced with different CARs upon overnight co-incubation with CD19-expressing target cells. FIG. 23B displays single cell cytokine analysis of 32 cytokines in CAR Tregs and CAR Teff cells. Note that not only there are differences across different CAR signaling modalities in the same cell type (e.g. 28z Tregs produce more cytokines in general than 41BBz or 28-TIGITz Tregs), but also there are marked differences between cell types transduced with the same CAR (e.g. 28z and 41BBz Teff are almost indistinguishable, whereas 28z Tregs produce cytokines to a much higher extent than 41BBz Tregs do). Specifically, both 41BB and TIGIT signaling suppress the production of the pro-inflammatory molecules Granzyme B, TNF-alpha, and IFN-gamma in CAR Tregs. This pattern is consistent between cytokine levels in the supernatant (FIG. 23A) and single cell intracellular cytokine analysis (FIG. 23B).

FIG. 24 shows that CD19 CAR Tregs remain stable after two weeks of in vitro activation and expansion by CD19-expressing target cells, as inferred from low Treg-specific demethylated region (TSDR) low methylation levels. Teff cells were used as a control for high TSDR methylation.

### DETAILED DESCRIPTION

Among the provided embodiments are chimeric antigen receptors (CARs). The recombinant receptors generally comprise an antigen-specific binding region, a transmembrane region, and an intracellular signaling region.

CAR-expressing regulatory T cells (Treg) provide an opportunity to generate antigen-specific Tregs for adoptive cell therapy. There are differences in function and signaling between Tregs and T effector cells (Teff). Accordingly, embodiments of the invention are directed to chimeric antigen receptors with a signaling region that maximizes the suppressive capacity and stability of Tregs for use in, but not limited to, antigen-specific cell therapies for autoimmune disorders, graft-versus-host disease, inflammatory diseases, and transplant rejection.

The invention is based, inter alia, on optimizing the intracellular signaling region of a chimeric antigen receptor for regulatory T cell function. This includes, but is not limited to, incorporating the signaling domains or combinations of signaling domains embodied herein, subsets of their sequences, domains derived from other species or viruses, non-immune signaling domains, synthetic domains or combinations thereof. These signaling architectures designed to maximize Treg function can be present in a CAR or any other chimeric receptor whose extracellular antigen recognition moiety includes, but is not limited to, a single chain antibody fragment (scFv) or another type of antibody-based molecule, or a functional non-T cell receptor, or any other antigen recognition molecule.

Regulatory T cells (Tregs): Tregs are important in the maintenance of immune cell homeostasis as evidenced by undesirable consequences of genetic or physical ablation of the Treg population. Treg cells generally maintain order in the immune system by enforcing a dominant negative regulation on other immune cells. Broadly classified into natural or adaptive (induced) Tregs; natural Tregs are CD4⁺CD25⁺ T-cells which develop, and emigrate from the thymus to play a role in immune homeostasis. Adaptive Tregs are non-regulatory CD4⁺ T-cells which acquire CD25 (IL-2R alpha) expression outside of the thymus, and may be induced by inflammation and disease processes, such as autoimmunity and cancer.

There is increasing evidence that Tregs acquire their function through a myriad of mechanisms that may include the secretion of immunosuppressive soluble factors such as IL-9, IL-10 and TGF beta, cell contact mediated regulation via the high affinity TCR and other costimulatory molecules such as CTLA-4, GITR, and cytolytic activity. Under the influence of TGF beta, adaptive Treg cells mature in peripheral sites, including mucosa-associated lymphoid tissue (MALT), from CD4⁺ Treg precursors, where they acquire the expression of markers typical of Tregs, including CD25, CTLA4 and GITR/AITR. Upon up-regulation of the transcription factor Foxp3, Treg cells begin their suppressive effect. This includes the secretion of cytokines including IL-10 and TGF beta which may induce cell-cycle arrest or apoptosis in effector T cells, and blocking co-stimulation and maturation of dendritic cells.

Tregs are hyporesponsive to TCR-mediated signaling, exhibiting low phosphorylation of CD3ζ, ERK, and AKT, among other downstream signaling molecules, when compared to Teff (7, 8). Generating a CAR with a subdued TCR signal component could be beneficial for CAR Treg engineering. CD3 possesses three immunoreceptor tyrosine-based activation motifs (ITAMs), while other CD3 subunits, CD3γ, CD3δ, and CD3ε, possess one ITAM (9).

In addition to TCR-mediated signaling, in some embodiments, to promote full activation, a component for generating a secondary or co-stimulatory signal is also included. In some embodiments, the intracellular signaling domain comprises a CD28 co-stimulatory domain for Treg development, maintenance, and function. Absence of CD28 in Tregs does not affect Treg cell number; however, these cells have lower levels of CTLA-4, PD-1, and CCR6, and may result in systemic autoimmunity characterized by prominent skin inflammation (10). In NOD mice, CD28 deficiency may lead to defects in Treg development and homeostasis and exacerbated type 1 diabetes (11, 12). Studies suggest that CD28 may function as an amplifier of TCR signaling; prolonged presence of antigen can sustain a functional T cell response in the absence of CD28 (13). CD28 contains a series of signaling motifs that can elicit intracellular phosphorylation cascades independent of TCR signals. CD28 tail motifs include an YMNM motif, which binds to the p85 subunit of PI3K, eliciting PI3K/Akt signaling, and a PYAP motif, which binds to FLNA, a regulator of cytoskeletal rearrangement, and the kinase LCK. In addition, both motifs bind the adaptor protein GRB2, which can bind Vav, which participates in various signaling complexes (14). A third motif present in the CD28 cytoplasmic domain is the PRRP motif, which binds the T cell-specific tyrosine kinase ITK and has been shown to be capable of inducing co-stimulation in murine primary T cells (17, 18).

As mentioned above, Tregs are hyporesponsive to TCR-mediated signaling when compared to Teff cells (7, 8). IL-2 signaling does not appear to trigger downstream targets of PI3K/Akt in Tregs, in contrast with Teff cells (19).

Tregs are capable of constitutively expressing a range of receptors not found in Teff cells at steady state. These include the inhibitory receptors CTLA4, PD1, TIM3, LAG3, and TIGIT, whose presence in Teff cells may signify dysfunction or exhaustion (22, 23). Without wishing to be bound by theory, including signaling motifs from these molecules may produce CARs that work optimally in Tregs and maximize their suppressive function. Tregs are capable of upregulating the expression levels of several tumor necrosis factor receptor (TNFR) superfamily members upon maturation in vivo, such as 41BB, TACI, HVEM, GITR, OX40, CD27, CD30, and TNFR2 (24, 25).

Accordingly, in certain embodiments, a chimeric antigen receptor (CAR) comprises an antigen specific binding domain, a spacer domain, a transmembrane domain, and an intracellular signaling region, the signaling region comprising a primary signaling domain, optionally derived from a CD3 chain domain, and a second signaling domain which is a costimulatory or inhibitory signaling domain of a protein selected from the group consisting of: CD28, ICOS, CTLA4, 41BB, CD27, CD30, CD132, OX-40, TACI, GITR, HVEM, TIM3, other TNFR superfamily members, and derivatives, mutants, variants, fragments and combinations thereof. In certain embodiments, the antigen specific binding domain comprises an antibody, a T cell receptor variable region, soluble T cell receptors, aptamer, nanobody, receptors, ligands, fragments or combinations thereof.

In certain embodiments, the primary signaling domain is or comprises the CD3 chain domain, wherein the CD3 chain is selected from the group consisting of: a CD3 zeta (CD3ζ) chain, a CD3 gamma (CD3γ) chain, a CD3 delta (CD3δ) chain, a CD3 epsilon (CD3ε) chain, derivatives, mutants, variants, fragments and combinations thereof. In certain embodies, the primary signaling domain optionally further comprises an Fc domain from the immunoglobulin superfamily, such as for example, FcγRI (CD64), FcγRIIA (CD32), FcγRIIB (CD32), FcγRIIIA (CD16a), FcγRIIIB (CD16b), FcαRI (CD89), FcεRI, FcεRII (CD23), Fcα, FcμR, derivatives, mutants, variants, fragments and combinations thereof. In certain embodiments, the Fc domain is an Fcγ domain, derivatives, mutants, variants, fragments and combinations thereof. As used herein, an “Fcγ domain” includes FcγRI (CD64), FcγRIIA (CD32), FcγRIIB (CD32), FcγRIIIA (CD16a), FcγRIIIB (CD16b) derivatives, mutants, variants, and fragments thereof.

In certain embodiments, a co-stimulatory signaling domain comprises CD28, ICOS, CTLA4, 41BB, CD27, CD30, derivatives, mutants, variants, fragments or combinations thereof. In certain embodiments, an inhibitory signaling domain comprises CTLA4, PD-1, TIM3, LAG3, TIGIT, mutants, variants, fragments or combinations thereof. In other embodiments, the costimulatory domain comprises CD28, 41BB, mutants or fragments thereof. In other embodiments, the costimulatory domain comprises ICOS, 41BB, mutants or fragments thereof. In other embodiments, the costimulatory domain comprises CTLA4, 41BB, mutants or fragments thereof.

In certain embodiments, an intracellular signaling region comprises (i) CD28, ICOS, CTLA4, 41BB or combinations thereof; (ii) at least one domain selected from TACI, HVEM, GITR, OX40, CD27, CD30; and (iii) a CD3 zeta (CD3ζ) chain, a CD3 gamma (CD3γ) chain, a CD3 delta (CD3δ) chain, a CD3 epsilon (CD3ε) chain, Fey or combinations thereof.

In certain embodiments, a chimeric antigen receptor comprises an antigen specific binding domain and at least one intracellular signaling region, the signaling region comprising (i) CD28; (ii) 41BB, TACI, HVEM, GITR, OX40, CD27 or CD30; and (iii) a CD3ζ chain and/or an Fcγ chain.

In certain embodiments, a chimeric antigen receptor comprises an antigen specific binding domain and at least one intracellular signaling region, the signaling region comprising (i) CD28; (ii) CTLA4, PD-1, TIM3, LAG3 or TIGIT; and (iii) a CD3ζ chain and/or an Fcγ chain.

In certain embodiments, a chimeric antigen receptor comprises an antigen specific binding domain and at least one intracellular signaling region, the signaling region comprising (i) CD28; (ii) CD132; and (iii) a CD3ζ chain and/or an Fcγ chain.

In certain embodiments, a chimeric antigen receptor comprises an antigen specific binding domain and at least one intracellular signaling region, the signaling region comprising (i) ICOS; (ii) 41BB; and (iii) a CD3 chain and/or an Fcγ chain.

In certain embodiments, a chimeric antigen receptor comprises an antigen specific binding domain and at least one intracellular signaling region, the signaling region comprising (i) CTLA4; (ii) 41BB; and (iii) a CD3ζ chain and/or an Fcγ chain.

In certain embodiments, a signaling region comprises (i) CD28, 41BB or a combination thereof; (ii) at least one domain selected from CTLA4, PD1, TIM3, LAG3, or TIGIT; (iii) a CD3 zeta (CD3ζ) chain, a CD3 gamma (CD3γ) chain, a CD3 delta (CD3δ) chain, a CD3 epsilon (CD3ε) chain, an Fcγ chain or combinations thereof.

In some embodiments, the CAR may also comprise a spacer domain situated between the antigen binding region and T cell plasma membrane. The spacer domain may include a sequence derived from IgG subclass IgG1, IgG4, IgD or CD8. In certain embodiments, the spacer domain comprises a CD28 motif. The spacer domain can have any length. In some embodiments, the spacer domain comprises 1 amino acid or 10 amino acids or 20 amino acids or 50 amino acids or 60 amino acids or 70 amino acids or 80 amino acids or 100 amino acids or 120 amino acids or 140 amino acids or 160 amino acids or 180 amino acids or 200 amino acids or 250 amino acids or 300 amino acids or any number therebetween.

In some embodiments, a CAR may further comprise a linker region. The linker may be rich in glycine, serine, and/or threonine for solubility. The linker region can connect to N-terminus of variable heavy (VH) chain with the C-terminus of the variable light (VL) chain or vice versa.

Antigen binding domain: Numerous antigen-binding domains are known in the art, including those based on the antigen binding site of an antibody, antibody mimetics, nanobodies, and T-cell receptor fragments. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder such as a DARPin; or a single-chain derived from a T-cell receptor. Accordingly, the antigen specific binding domain includes, without limitation, an antibody, a T cell receptor fragment, a soluble T cell receptor, nanobody, aptamer, syn/notch recognition domain/effector domain pair, receptors, fragments or combinations thereof. In certain embodiments, the antigen specific binding domain is a T cell variable region fragments. In other embodiments, the antigen specific binding domain is an antibody or fragment thereof. The CAR can include single chains of T cell receptors and antibodies. In certain embodiments, the antigen binding domain is a single chain fragment is a single chain variable fragment (scFv).

In certain embodiments, the antigen binding domain is or comprises an antibody or antibody fragment. In certain embodiments, the antibodies are human antibodies, including any known to bind a targeting molecule. The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding domain is a humanized antibody or fragments thereof. A “humanized” antibody is an antibody in which all or substantially all CDR amino acid residues are derived from non-human CDRs and all or substantially all framework region (FR) amino acid residues are derived from human FRs. A humanized antibody optionally may include at least a portion of an antibody constant region derived from a human antibody. A “humanized form” of a non-human antibody, refers to a variant of the non-human antibody that has undergone humanization, in some cases to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain”, when used in reference to an antibody, such as an antibody fragment, refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

Regulatory T cells: In general, T regulatory cells have been identified as a CD4⁺CD25⁺ T cell population capable of suppressing an immune response. The identification of Foxp3 as a “master-regulator” of Tregs helped define Tregs as a distinct T cell lineage. The identification of additional antigenic markers on the surface of Tregs has enabled identification and FACS sorting of viable Tregs to greater purity, resulting in a more highly-enriched and suppressive Treg population. In addition to CD4 and CD25, both mouse and human Tregs express GITR/AITR, CTLA-4, and express low levels of CD127 (IL-7Ra). Moreover, Tregs can exist in different states which can be identified based on their expression of surface markers. Tregs which develop in the thymus from CD4⁺ thymocytes are known as “natural” Tregs, however Tregs can also be induced in the periphery from naïve CD4⁺ T cells in response to low-dose engagement of the TCR, TGF beta and IL-2. These “induced” Tregs secrete the immunosuppressive cytokine IL-10. The phenotype of Tregs changes again as they become activated, and markers including GARP in mouse and human, CD45RA in human, and CD103 in mouse have been shown to be useful for the identification of activated Tregs.

Accordingly, in certain embodiments, an isolated T cell is modified to express a chimeric antigen receptor (CAR) comprising an antigen specific binding domain, a spacer domain, a transmembrane domain, and an intracellular signaling region, the signaling region comprising a primary signaling domain, optionally derived from a CD3 chain domain, and a second signaling domain which is a costimulatory or inhibitory signaling domain of a protein selected from the group consisting of: CD28, ICOS, CTLA4, 41BB, CD27, CD30, CD132, OX-40, TACI, GITR, HVEM, TIM3, other TNFR superfamily members, and derivatives, mutants, variants, fragments and combinations thereof.

In certain embodiments, the primary signaling domain is or comprises a CD3 chain domain, wherein the CD3 chain is selected from the group consisting of: a CD3 zeta (CD3ζ) chain, a CD3 gamma (CD3γ) chain, a CD3 delta (CD3δ) chain, a CD3 epsilon (CD3ε) chain, derivatives, mutants, variants, fragments and combinations thereof.

In certain embodiments, a Treg costimulatory signaling domain comprises CD28, ICOS, CTLA4, 41BB, CD27, CD30, mutants, variants, fragments or combinations thereof. In certain embodiments, the Treg inhibitory signaling domain comprises CTLA4, PD-1, TIM3, LAG3, TIGIT, mutants, variants, fragments or combinations thereof. In other embodiments, the costimulatory signaling domain comprises CD28, 41BB, mutants or fragments thereof. In other embodiments, the costimulatory signaling domain comprises ICOS, 41BB, mutants or fragments thereof. In other embodiments, the costimulatory signaling domain comprises CTLA4, 41BB, mutants or fragments thereof.

In certain embodiments, the costimulatory signaling domain comprises (i) CD28, ICOS, CTLA4, 41BB or combinations thereof; (ii) at least one domain selected from TACI, HVEM, GITR, OX40, CD27, CD30; and (iii) a CD3 zeta (CD3ζ) chain, a CD3 gamma (CD3γ) chain, a CD3 delta (CD3δ) chain, a CD3 epsilon (CD3ε) chain, or combinations thereof.

In certain embodiments, the CAR comprises an antigen specific binding domain and at least one signaling region, the signaling region comprising (i) CD28; (ii) 41BB, TACI, HVEM, GITR, OX40, CD27 or CD30; and (iii) a CD3ζ chain and/or Fcγ chain.

In certain embodiments, the CAR comprises an antigen specific binding domain and at least one signaling region, the signaling region comprising (i) CD28; (ii) CTLA4, PD-1, TIM3, LAG3 or TIGIT; and (iii) a CD3ζ chain and/or Fcγ chain.

In certain embodiments, the CAR comprises an antigen specific binding domain and at least one signaling region, the signaling region comprising (i) CD28; (ii) CD132; and (iii) a CD3ζ chain and/or Fcγ chain.

In certain embodiments, the CAR comprises an antigen specific binding domain and at least one signaling region, the signaling region comprising (i) ICOS; (ii) 41BB; and (iii) a CD3ζ chain and/or Fcγ chain.

In certain embodiments, the CAR comprises an antigen specific binding domain and at least one signaling region, the signaling region comprising (i) CTLA4; (ii) 41BB; and (iii) a CD3ζ chain and/or Fcγ chain.

In certain embodiments, a signaling region comprises (i) CD28, 41BB or a combination thereof; (ii) at least one domain selected from CTLA4, PD1, TIM3, LAG3, or TIGIT; and (iii) a CD3 zeta (CD3ζ) chain, a CD3 gamma (CD3γ) chain, a CD3 delta (CD3δ) chain, a CD3 epsilon (CD3ε) chain, or combinations thereof.

In certain embodiments, the Treg cell is CD4⁺CD25⁺ CD127⁻, FOXP3⁺ and Helios⁺.

Methods of Treatment

Also provided are methods of treatment. In certain embodiments, a method of treating a subject suffering from an autoimmune or inflammatory disease or disorder, comprises isolating and separating CD4⁺ T regulatory cells (Tregs) from a subject's biological sample, wherein the Treg cells are CD4⁺CD25⁺CD127⁻; contacting the Treg cells with an expression vector encoding a chimeric antigen receptor (CAR) which specifically binds to an antigen associated with an autoimmune response and/or suppresses an effector T cell (Teff) or inflammatory immune response; stimulating the transduced Treg with a specific antigen to obtain a therapeutically effective number of antigen-specific Treg cells; and, reinfusing the Treg into the subject.

A similar protocol can be effected in treating a subject suffering from graft-versus-host disease (GVHD) or in a subject who has received or will be receiving an organ transplantation, skin graft etc.

In certain embodiments, the Treg cells are autologous cells. CAR-T cells may be generated from any suitable source of T cells known in the art including, but not limited to, T cells collected from a subject. The subject may be a patient with an autoimmune disease in need of CAR-T cell therapy or a subject of the same species as the subject with the autoimmune disease in need of CAR-T cell therapy. The collected T cells may be expanded ex vivo using methods commonly known in the art before transduction with a CAR to generate a CAR-T cell.

Methods for CAR design, delivery and expression in T cells, and the manufacturing of clinical-grade CAR-T cell populations are known in the art. See, for example, Lee et al., Clin. Cancer Res. (2012) 18(10):2780-90, hereby incorporated by reference in its entirety. For example, the engineered CARs may be introduced into T cells using retroviruses, which efficiently and stably integrate a nucleic acid sequence encoding the chimeric antigen receptor into the target cell genome. An exemplary method is described in the Examples section which follows.

Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cash, Cas6e, Cas6f, Cas7, Cas8a1, Cas8a2, Cas8b, Cas8c, Cas9, Cas10, Cas10d, Cas12a (Cpf1), Cas13a (C2c2), Cas13b, Cas13d, CasF, CasG, CasH, Csy1, Csy2, Csy3, Cse1 (or CasA), Cse2 (or CasB), Cse3 (or CasE), CasX, CasY, Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.).

The CAR-T cells, once they have been expanded ex vivo in response to, for example, an autoimmune disease antigen, can be reinfused into the subject in a therapeutically effective amount. The term “therapeutically effective amount” as used herein means the amount of CAR T cells when administered to a mammal, in particular a human, in need of such treatment, is sufficient to treat autoimmune diseases, or prevent organ rejection etc.

The precise amount of CART cells to be administered can be determined by a physician with consideration of individual differences in age, weight, extent of disease and condition of the subject.

Administration of T cell therapies may be defined by number of total cells per infusion or number of cells per kilogram of body weight, especially for pediatric patients. As T cells replicate and expand after transfer, the administered cell dose may not resemble the final steady-state number of cells. In an embodiment, a pharmaceutical composition comprising the CAR T cells of the present invention may be administered at a dosage of 10⁴ to 10¹⁰ total cells. In another embodiment, a pharmaceutical composition comprising the CAR T cells of the present invention may be administered at a dosage of 10³ to 10⁸ cells/kg body weight, including all integer values within those ranges.

Compositions comprising the CAR T cells of the present invention may also be administered multiple times at these dosages. The cells can be administered by using infusion techniques that are known in the art (see, for example, Rosenberg et al., 1988, New England Journal of Medicine, 319:1676). The optimal dosage and treatment regimen for a particular subject can be determined by one skilled in the art by monitoring the patient for signs of disease and adjusting the treatment accordingly.

In certain embodiments, administration of any of the compositions embodied herein, for the treatment of, for example, an autoimmune or inflammatory disease, can be combined with other cell-based therapies, for example, stem cells, antigen presenting cells, pancreatic islets etc.

The composition of the present invention may be prepared in a manner known in the art and in a manner suitable for parenteral administration to mammals, particularly humans, comprising a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the invention may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the CAR T cells. In general, the compositions are prepared by uniformly and intimately bringing into association any active ingredients with liquid carriers.

In an embodiment, the composition is suitable for parenteral administration. In another embodiment, the composition is suitable for intravenous administration.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

The invention also contemplates the combination of the composition of the present invention with other drugs and/or in addition to other treatment regimens or modalities such as surgery. When the composition of the present invention is used in combination with known therapeutic agents the combination may be administered either in sequence (either continuously or broken up by periods of no treatment) or concurrently or as an admixture. In the case of, for example, autoimmune diseases, treatment comprises administering to the subject the compositions embodied herein, e.g. autologous T cells transduced or contacted with a CAR embodied herein and one or more anti-inflammatory agents and/or therapeutic agents. The anti-inflammatory agents comprise one or more antibodies which specifically bind to pro-inflammatory cytokines, e.g. pro-inflammatory cytokines such as IL-1, TNF, IL-6, GM-CSF, and IFN-γ. In certain embodiments, the antibodies are anti-TNFα, anti-IL-6 or combinations thereof. In certain embodiments, one or more agents, other than antibodies can be administered which decrease pro-inflammatory cytokines, e.g. non-steroidal anti-inflammatory drugs (NSAIDs). Any combination of antibodies and one or more agents can be administered which decrease pro-inflammatory cytokines.

Treatment in combination is also contemplated to encompass the treatment with either the composition of the invention followed by a known treatment, or treatment with a known agent followed by treatment with the composition of the invention, for example, as maintenance therapy. For example, in the treatment of autoimmune diseases, excessive and prolonged activation of immune cells, such as T and B lymphocytes, and overexpression of the master pro-inflammatory cytokine tumor necrosis factor alpha (TNF), together with other mediators such as interleukin-6 (IL-6), interleukin-1 (IL-1), and interferon gamma (IFN-γ), play a central role in the pathogenesis of autoimmune inflammatory responses in rheumatoid arthritis (RA), inflammatory bowel disease (IBD), Crohn's disease (CD), and ankylosing spondylitis (AS).

Non-steroidal anti-inflammatory drugs (NSAIDs), glucocorticoids, disease-modifying anti-rheumatic drugs (DMARDs) are traditionally used in the treatment of autoimmune inflammatory diseases. NSAIDs and glucocorticoids are effective in the alleviation of pain and inhibition of inflammation, while DMARDs have the capacity of reducing tissue and organ damage caused by inflammatory responses. More recently, treatment for RA and other autoimmune diseases has been revolutionized with the discovery that TNF is critically important in the development of the diseases. Anti-TNF biologics (such as infliximab, adalimumab, etanercept, golimumab, and certolizumab pepol) have markedly improved the outcome of the management of autoimmune inflammatory diseases.

Non-steroidal anti-inflammatory drugs have the analgesic, antipyretic, and anti-inflammatory effect, frequently used for the treatment of conditions like arthritis and headaches. NSAIDs relieve pain through blocking cyclooxygenase (COX) enzymes. COX promotes the production of prostaglandins, a mediator which causes inflammation and pain. Although NSAIDs have different chemical structures, all of them have the similar therapeutic effect, e.g., inhibition of autoimmune inflammatory responses. In general, NSAIDs can be divided into two broad categories: traditional non-selective NSAIDs and selective cyclooxygenase-2 (COX-2) inhibitors (For a review, see, P. Li et al. Front Pharmacol. (2017) 8:460).

In addition to anti-TNF agents, the biologics targeting other proinflammatory cytokines or immune competent molecules have also been extensively studied and actively developed. For example, abatacept, a fully humanized fusion protein of extracellular domain of CTLA-4 and Fc fraction of IgG1, has been approved for the RA patients with inadequate response to anti-TNF therapy. The major immunological mechanism of abatacept is selective inhibition of co-stimulation pathway (CD80 and CD86) and activation of T cells. Tocilizumab, a humanized anti-IL-6 receptor monoclonal antibody was approved for RA patients intolerant to DMARDs and/or anti-TNF biologics. This therapeutic mAb blocks the transmembrane signaling of IL-6 through binding with soluble and membrane forms of IL-6 receptor. Biological drugs targeting IL-1 (anakinra), Th1 immune responses (IL-12/IL-23, ustekinumab), Th17 immune responses (IL-17, secukinumab) and CD20 (rituximab) have also been approved for the treatment of autoimmune diseases (For a review see, P. Li et al. Front Pharmacol. 2017; 8: 460).

Methods for Isolation of Cells

Any number of methods known in the art can be used to isolate cells, for transduction with any number of CARs embodied herein, such as Tregs, or any other cell type that may be used in carrying out the treatment of a subject. Thus, also provided are various other genetically engineered cells expressing the chimeric antigen receptors e.g., CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4⁺ and/or of CD8⁺ T cells are naive T (T_{N}) cells, effector T cells (T_{EFF}), memory T cells and sub-types thereof, such as stem cell memory T (T_{SCMX}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and induced regulatory T (Treg) cells, helper T cells, such as T_{H}1 cells, T_{H}2 cells, T_{H}3 cells, T_{H}17 cells, T_{H}9 cells, T_{H}22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

Exemplary methods of isolating cells and engineering these cells with a CAR are described in the Examples section which follows.

In some embodiments, preparation of the engineered cells includes one or more culture and/or preparation steps. The cells for introduction of the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In certain embodiments, a biological sample is obtained from one or more sources comprising: autologous, allogeneic, haplotype matched, haplotype mismatched, haplo-identical, xenogeneic, cell lines or combinations thereof.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be useful where no antibody is available that specifically identifies a cell type in a heterogeneous population, such that separation is carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. For example, in some aspects, specific subpopulations of T cells, such as cells positive or expressing one or more markers, e.g., CD4⁺CD25⁺, FOXP3⁺ and Helios⁺.

T cells, are isolated by positive or negative selection techniques. For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker 1″) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD 14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some aspects, a CD4 expression-based selection step is used to generate the CD4⁺ cell population or sub-population, such that both the positive and negative fractions from the CD4-based separation are retained and used in subsequent steps of the methods, optionally following one or more further positive or negative selection steps.

In one example, a sample of PBMCs or other white blood cell sample is subjected to selection of CD4⁺ cells, where both the negative and positive fractions are retained. The negative fraction then is subjected to negative selection based on expression of, for example, CD 14 and CD45RA, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naïve CD4⁺ T lymphocytes are CD45RO⁺, CD45RA⁺, CD62L⁺, or CD4⁺ T cells. In some embodiments, central memory CD4⁺ cells are CD62L⁺ and CD45RO⁺.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen, U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto are capable of being attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9):651-60, Terakura et al. (2012) Blood 1:72-82, and Wang et al. (2012) Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-73; and Godin et al. (2008) J Biophoton. 1(5):355-76. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al.; Klebanoff et al. (2012) J Immunother. 35(9): 651-60, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25° C., generally at least about 30° C., and generally at or about 37° C. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1: Cellular Signaling Domains Engineering in Chimeric Antigen Receptors

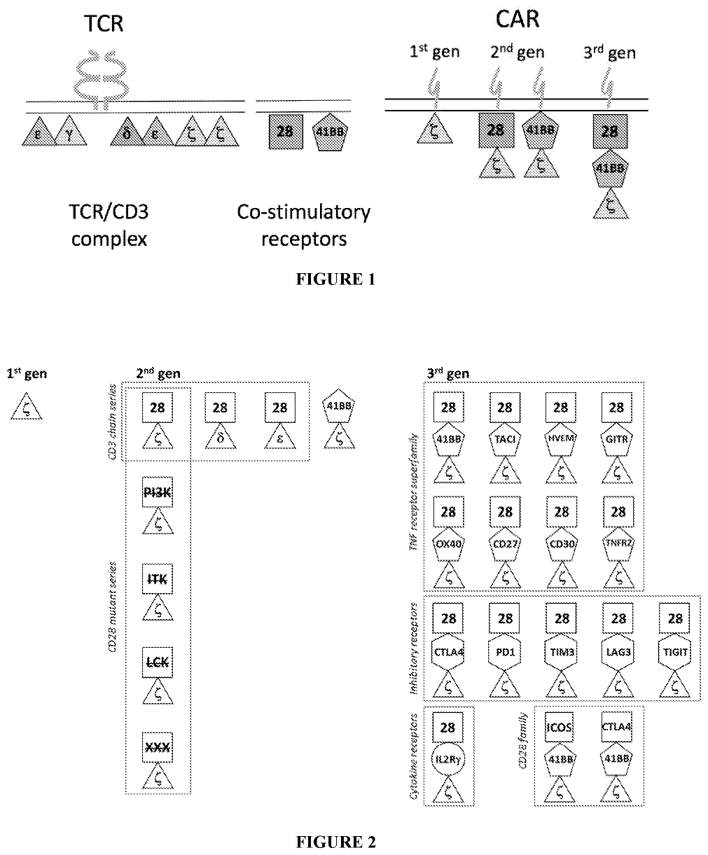

It was hypothesized that CAR-mediated signaling is determined by the identity of its signaling domains and has a measurable impact on CAR Treg function. Modifying the signaling component of a CAR by introducing one or more endodomains significantly impacts CAR-mediated signaling. CAR signaling domains were designed herein to combine multiple signaling modules in one single molecule (FIG. 1).

A. CAR Library

A library of 18 constructs focused on dissecting signaling of the traditional 28-ζ CAR were generated, which can be divided into two main groups. In one group, the CD3zeta chain was replaced with CD3gamma, CD3delta, or CD3epsilon downstream of CD28. In another group, the signaling domain of CD28 was systematically mutated, creating constructs with point mutations ablating the canonical motifs binding to PI3K, ITK, and LCK, either individually or in combinations. In addition to these two groups, there are also some constructs where mutations in CD28 were combined with alternative CD3 chains in the same CAR.

Another library was generated and termed “exotic” CARs, containing various signaling domains inspired by Treg biology upstream of CD3ζ. Initially, CD28z and 41BBz were used, followed by “exotic” CARs divided into 4 groups: CD28 family, where CD28 was replaced by either ICOS or CTLA4 upstream of 41BBz; TNFR family, where TACI, HVEM, GITR, OX40, CD27, or CD30 were inserted between CD28 and CD3ζ; inhibitory receptors, where CTLA4, PD1, TIM3, LAG3, or TIGIT were inserted between CD28 and CD3ζ; common gamma chain, where the common gamma chain/IL-2Rγ/CD132 was inserted between CD28 and CD3ζ. A schematic of all the CAR constructs generated are shown in FIG. 2.

B. CAR Delivery and In Vitro Assay of CAR Function

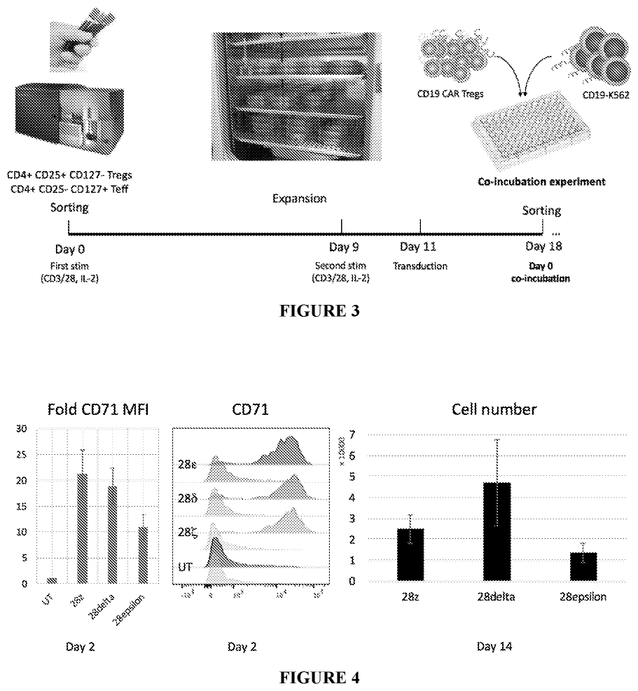

Second generation CD19 CARs containing CD3ζ, CD3γ, CD3δ, or CD3ε were introduced downstream of CD28 in peripheral blood-derived human Tregs via lentiviral transduction. The resulting CAR Tregs were stimulated with CD19-expressing K562 cells in vitro and evaluated with regards to 1) early activation by the expression of CD71, ICOS, and CD25; 2) proliferation and expansion on day 14, 3) stability by FOXP3 expression and Treg-specific demethylated region (TSDR); and 4) suppression. To examine each of the first three properties in vitro, a co-culture system was established where CAR Tregs were stimulated via their CAR by irradiated CD19-K562 cells (FIG. 3).

C. CAR Treg-Mediated In Vivo Suppression

NSG mice were used to measure the efficacy of human Tregs transduced with the various CD19 CARs described above. 8-12-week-old NSG mice were injected subcutaneously with luciferase-labeled CD19-K562 cells. Ten days later, total CD19 CAR-expressing T cells were injected intravenously (retro-orbital route) either with or without CD19 CAR-expressing Tregs. CD19-28z CAR-expressing T cells led to tumor rejection (as assessed by luciferase activity and tumor volume) within 2 weeks; CD19-28z CAR-expressing Tregs prevented this rejection.

D. Signal 1 Engineering

Activated Tregs display quantitative increases in the surface expression of molecules present in steady state, such as CD25, ICOS, and CTLA4 (26). Flow cytometry was used to monitor cell cultures at different time points to assess 1) early activation, computed as changes in the mean fluorescence intensity (MFI) of CD25, CD71, and ICOS, 2) proliferation, by normalizing the number of events to a known amount of counting beads, and 3) stability, by measuring FOXP3 protein expression levels. Functional Treg markers, such as, CTLA4, were also monitored. Interestingly, preliminary data indicate that CD3ε may be incompatible with CAR Treg expansion (FIG. 4).

E. CD28 Engineering

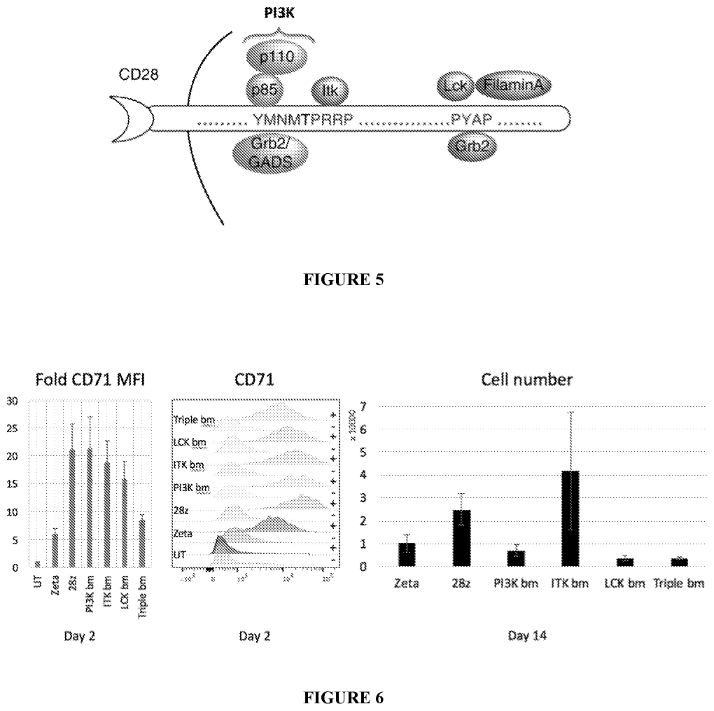

CD28 mutant CARs constructs featuring individual or combined mutations in each one of the canonical CD28 motifs upstream of CD3ζ were introduced in primary human Tregs and their assessment is carried out as described above. The motifs mutated were YMNM (PI3K binding), PRRP (ITK binding) and PYAP (LCK binding) (FIGS. 5 and 6).

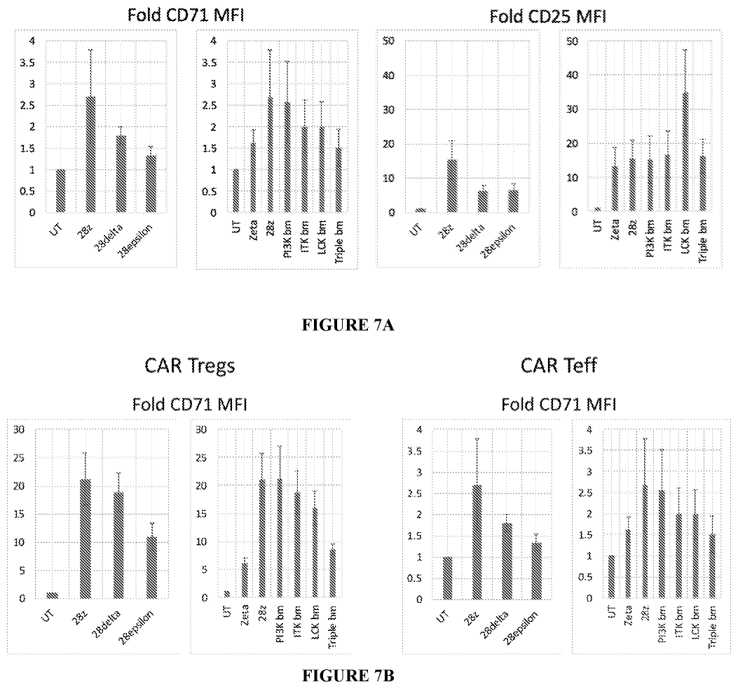

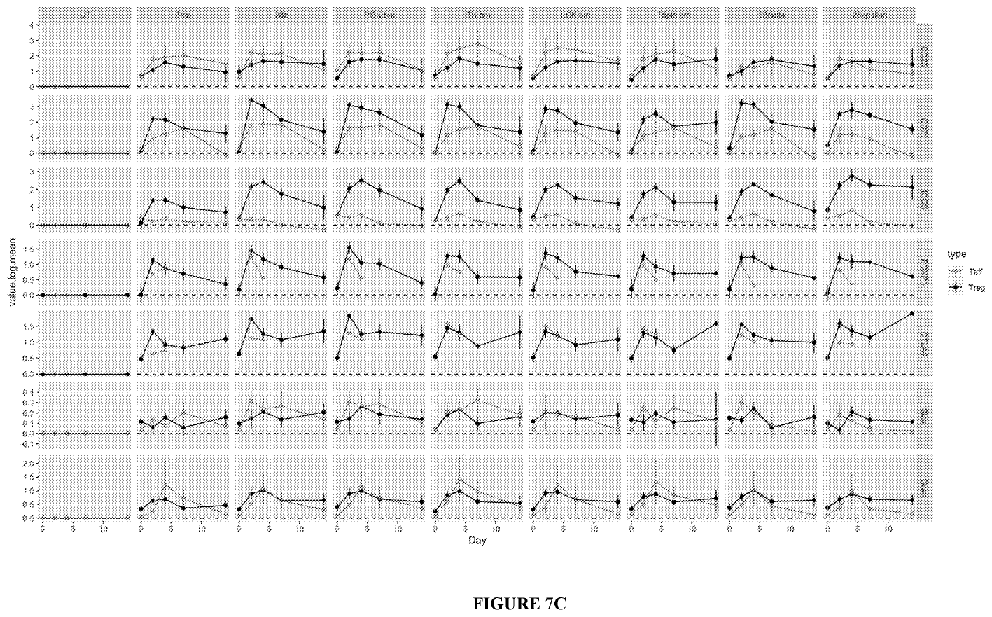

In Teff cells transduced with the same CD19 CAR constructs, the pattern of CD71 upregulation was similar to that of CD19 CAR-expressing Tregs (FIGS. 7A, 7B, 7C). However, measuring CD25 levels in CD19 CAR-expressing Teff (CD25 is constitutively highly expressed in Tregs) revealed a trend towards higher CD25 expression in CD19 CAR-expressing Teff cells harboring a CD19 CAR with a mutant CD28 domain containing a defective LCK binding motif (FIGS. 7A, 7B, 7C).

The data suggests that swapping the CD3 chain of the CAR affects CAR Treg expansion, and mutating the PI3K binding site of CD28 did not significantly alter early activation in either CAR Tregs or CAR Teff cells.

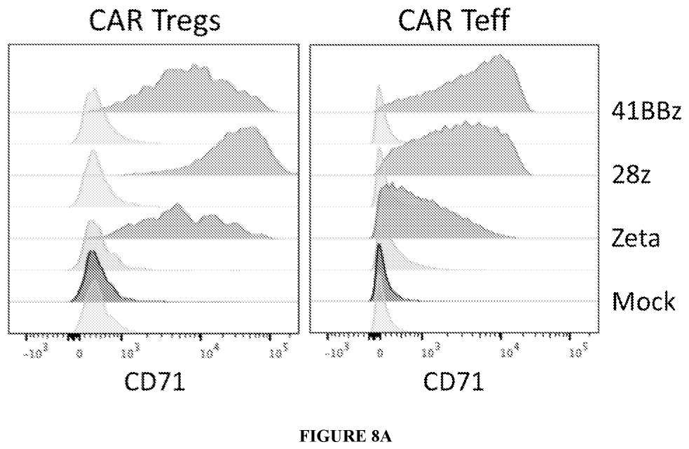

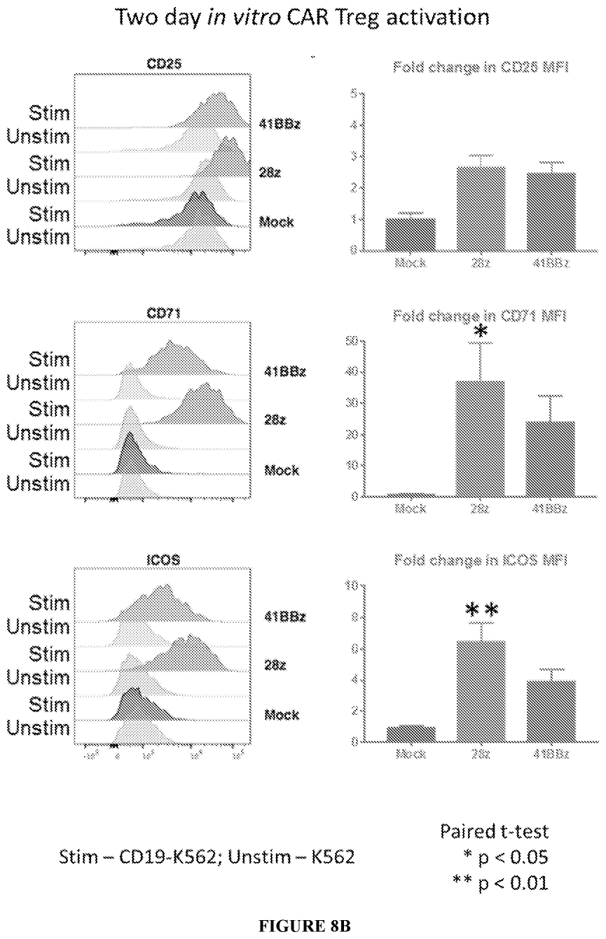

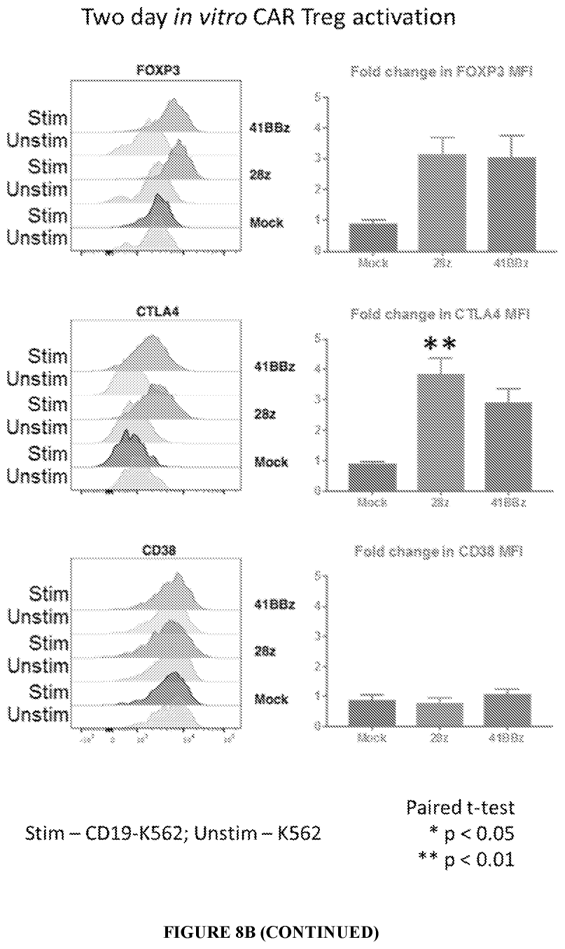

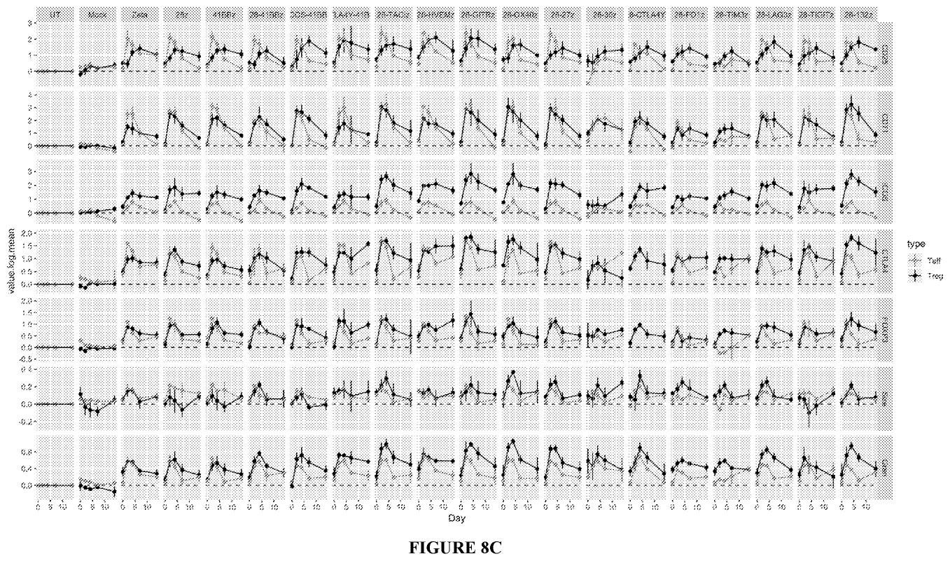

CD28 versus 41BB: Early activation of CAR Tregs and CAR Teff with constructs encoding signaling region of 28z or 41BBz was examined. (FIGS. 8A, 8B, 7C). As shown in FIGS. 8A, 8B, 7C, CD19 28z CAR-expressing Tregs upregulate CD71 to higher levels than their 41BBz counterparts. This effect was not observed in CD19 CAR-expressing Teff cells.

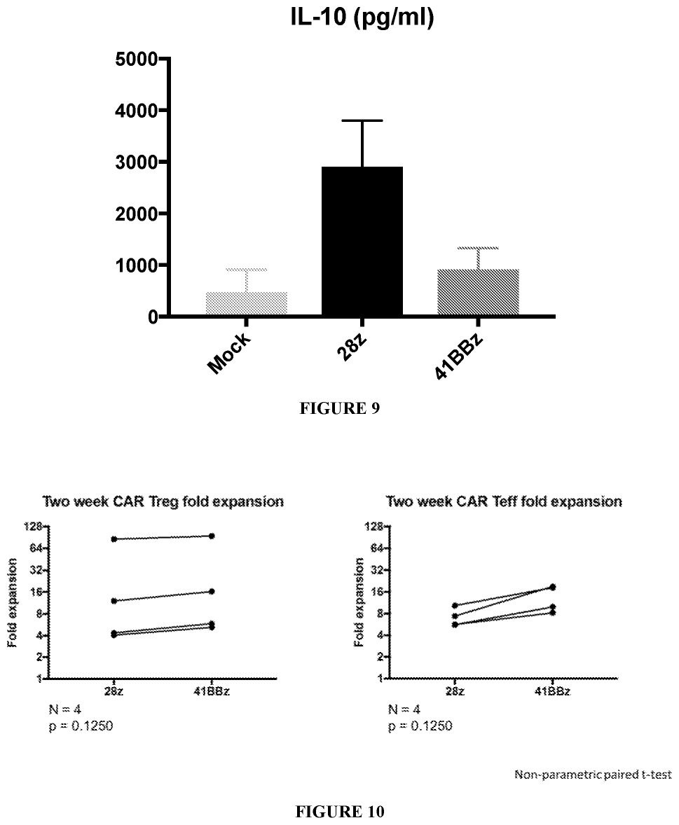

A hallmark of Tregs is the production of suppressive cytokines, including IL-10. Strikingly, as shown in FIG. 9, CD19 28z CAR-expressing Tregs produce IL-10 upon CAR activation with irradiated CD19-K562 cells for 2 days, whereas 41BBz CAR Tregs do not (FIG. 9).

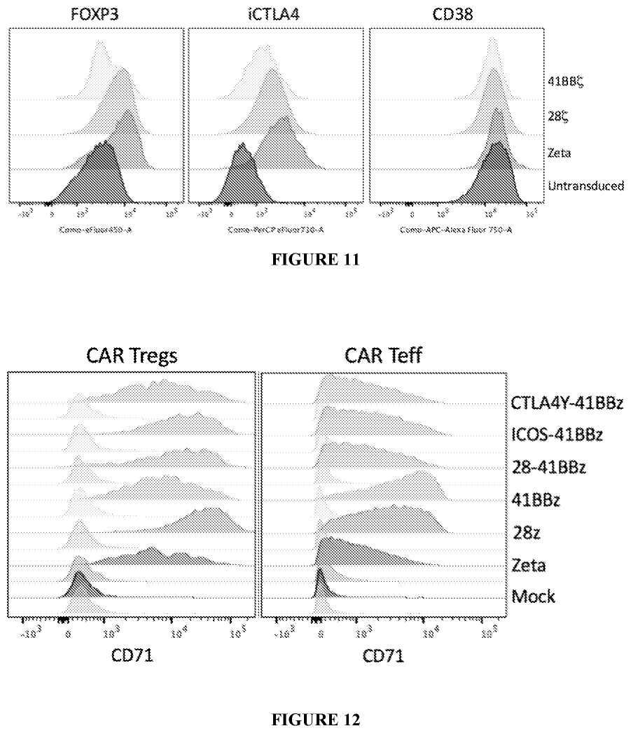

In addition, 41BBz failed to support CAR-expressing Treg expansion in vitro, even though it led to robust proliferation of CAR Teff cells (FIG. 10), and displayed lower FOXP3 and CTLA4 levels on Day 14 post CAR activation (FIG. 11). Those data, combined with the lack of IL-10 production, suggest that 41BBz CAR Tregs are poor suppressors.

These observations are interesting from a translational standpoint, because total PBMC or T cell preparations contain Tregs and if those are transduced with CAR together with the effector T cells, they could hamper anti-tumor activity of a given CAR T preparation. These data suggest that it could be beneficial to use 41BBz CAR for CAR T cell tumor therapy, as any CAR Tregs in the preparation may not expand properly or secrete suppressive cytokines.

F. Exotic CARS—CD28 Family

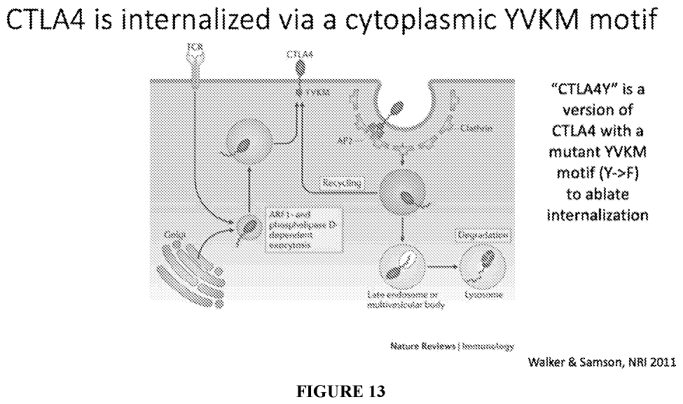

CD28 is part of a family of three closely related molecules: CD28, ICOS, CTLA4, CD28 and ICOS are co-stimulatory domains, whereas CTLA4 is an inhibitory receptor. CD28 expression is capable of supporting Treg development and homeostasis, ICOS expression has been associated with high suppressive capacity in human Tregs, and CTLA4 can be constitutively expressed in Tegs and is capable of supporting their suppressive function. CD19 28-41BBz CAR was tested together with CD19 ICOS-41BBz CAR and CD19 CTLA4Y-41BBz CAR in Tregs and Teff cells (FIG. 12). The CTLA4 domain which was used was mutated in the tyrosine of the YVKM motif (to FVKM) to prevent endocytosis of the CAR. This mutated CTLA4 domain was referred to as “CTLA4Y” (FIG. 13).

Results from the experiments indicate that the 3^{rd} generation CARs 28-41BBz, ICOS-41BBz, and CTLA4Y-41BBz result in a lesser degree of early activation than 28z CAR in both CAR-expressing Tregs and CAR-expressing Teff cells. Interestingly, 28-41BBz CAR-expressing Tregs yielded CD71 levels between those of 28z and 41BBz in CAR-expressing Tregs, and CTLA4Y-41BBz is comparable to 41BBz in CAR-expressing Tregs (FIG. 12).

G. Exotic CARs—TNFR Family

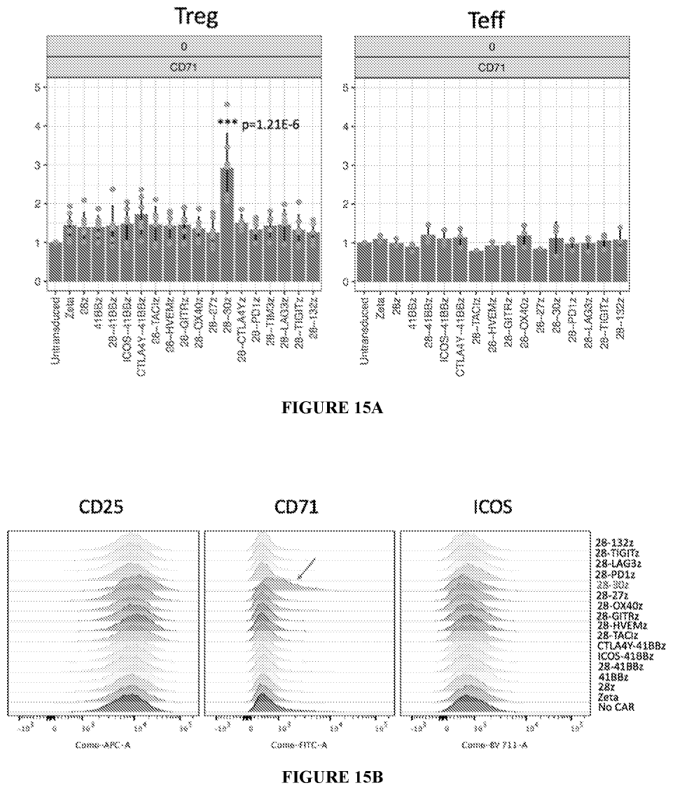

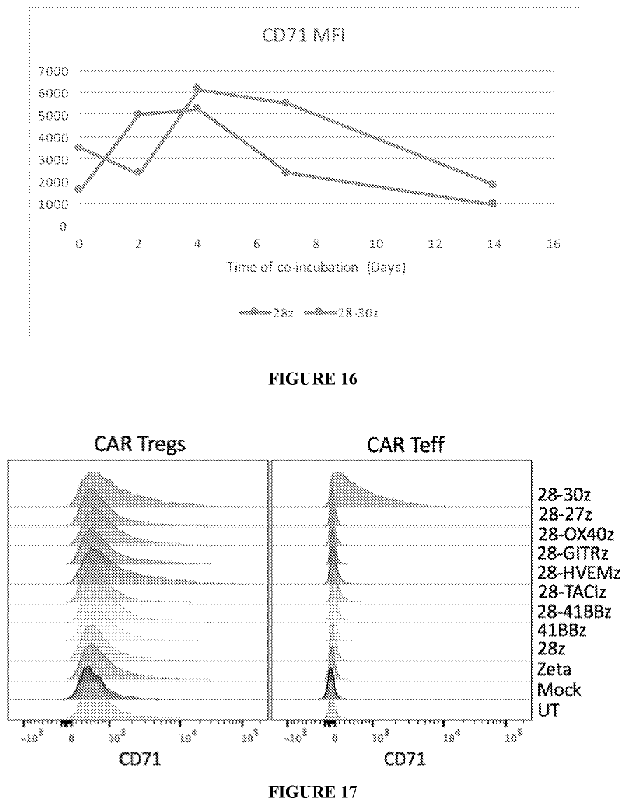

Analysis of early activation markers, such as CD71, revealed significant differences in some CAR constructs (FIG. 14). As shown in FIG. 14, 28-HVEMz and 28-30z CAR leads to a weaker upregulation of CD71 than 28z by Day 2 of co-incubation in both CAR-expressing Tregs and CAR-expressing Teff cells. Intriguingly, 28-30z CAR induced CD71 upregulation before co-incubation with CD19-K562 specifically in CAR-expressing Tregs, providing evidence for potential tonic signaling (FIG. 15). In fact, 28-30z CAR-expressing Treg and CAR-expressing Teff cells showed a delayed activation kinetics as compared with 28z (FIG. 16, 17). The 28-30z CAR would thus be useful in any context where delayed (CAR) T cell activation is desired.

H. Exotic CARs—Inhibitory Receptor

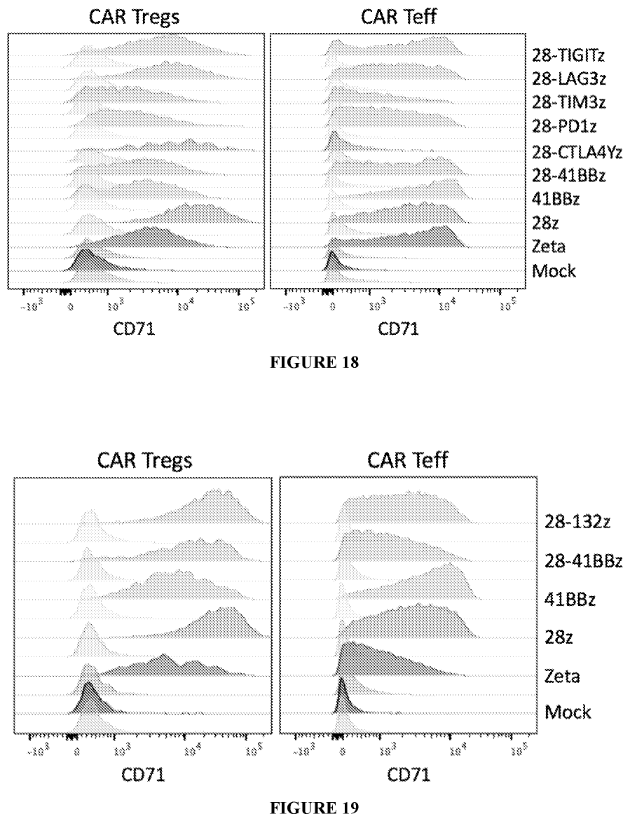

Tregs constitutively express inhibitory receptors, whereas Teff cells only express these in a state of exhaustion. It was hypothesized that including an inhibitory receptor domain in the CAR signaling region might lead to specific CAR-mediated early activation of Tregs (but not of Teff cells). This phenomenon was observed with one domain: CTLA4Y. 28-CTLA4Yz CAR leads to CD71 upregulation in CAR-expressing Tregs, but not in CAR-expressing Teff after two days of co-incubation with CD19-K562 cells (FIG. 18).

I. Exotic CARs—Common Gamma Chain

Cytokine signaling is capable of supporting activation and expansion of T cells. IL-2 supports the growth of both Tregs and Teff cells. Tregs do not produce IL-2, and are therefore dependent on the presence of exogenous IL-2. It was thus hypothesized that including elements of IL-2 signaling in a CAR would benefit CAR Treg vitality and function. IL2Rγ (CD132, common gamma chain) domain was incorporated downstream of CD28. Similar early activation to 28z in both CAR-expressing Tregs and CAR-expressing Teff was observed (FIG. 19).

Two chains, beta (CD122) and gamma (CD132) are capable of supporting signal transduction for the IL2 receptor. Future experiments include constructing a CAR with a CD122 signaling domain. The current data set suggested a benefit in using cytokine receptor signaling in CAR Tregs and established that it is at least not detrimental to CAR function in Tregs.

J. Exotic CARs

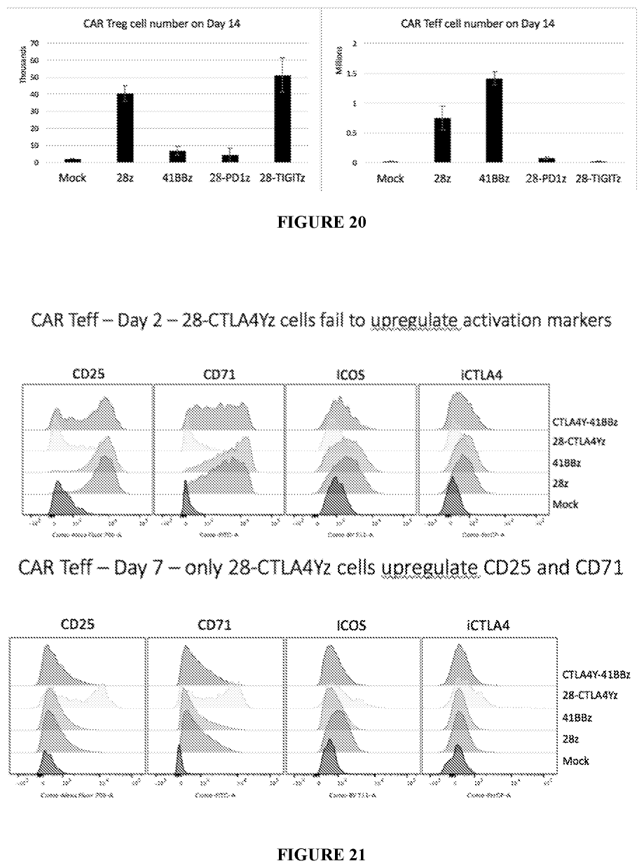

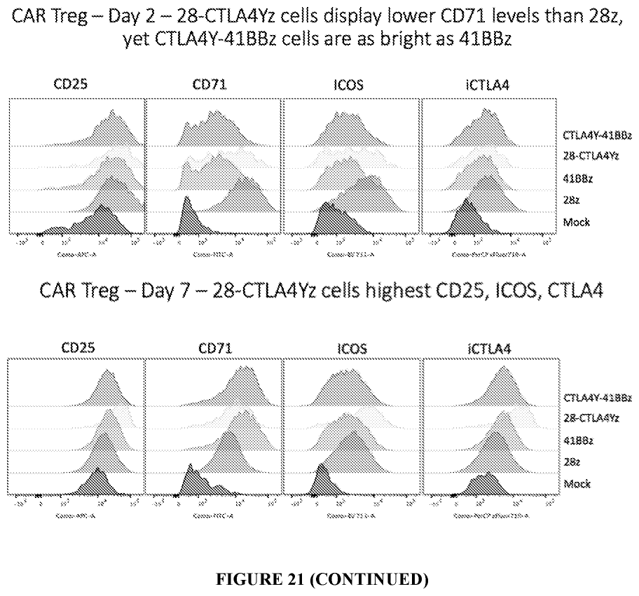

In addition to the unique pattern of CAR-mediated activation by 28-30z and 28-CTLA4Yz, the data showed CARs that preferentially lad to the expansion of either CAR-expressing Tregs or CAR-expressing Teff cells (FIG. 20). Tregs upregulated TNFR members (e.g. 41BB) upon maturation simultaneously with their constitutive expression of inhibitory receptors (e.g. CTLA4), leading some to suggest that TNFR upregulation with positive signaling partly compensated from negative signaling from inhibitory receptors. The same co-expression was observed in tumor infiltrating T cells. Interestingly, both in CAR-expressing Tregs and in CAR-expressing Teff, including CTLA4 signaling in the presence of CD28 (28-CTLA4Yz CAR) ablated early activation, but led to late expression of activation markers on Day 14, whereas including CTLA4 signaling in the presence of 41BB (CTLA4Y-41BBz CAR) did not lead to any significant difference from 41BBz alone CAR, suggesting a compensatory role of 41BB signaling (FIG. 21).

Finally, TIGIT signaling (28-TIGITz CAR) lea to expansion of CAR Tregs, but not of CAR Teff (FIG. 20). This observation was important from a translational standpoint, because even if a Treg preparation has some Teff contamination, which upon transduction would yield some CAR Teff that could be harmful, the 28-TIGITz seemed to selectively promote the expansion of Tregs. Altogether, these experiments demonstrated that not all TNFR domains are equal and not all inhibitory receptor are equal in the context of a CAR. Their specific properties in a CAR are not predicable by prior art.

K In Vivo CAR Treg-Mediated Suppression

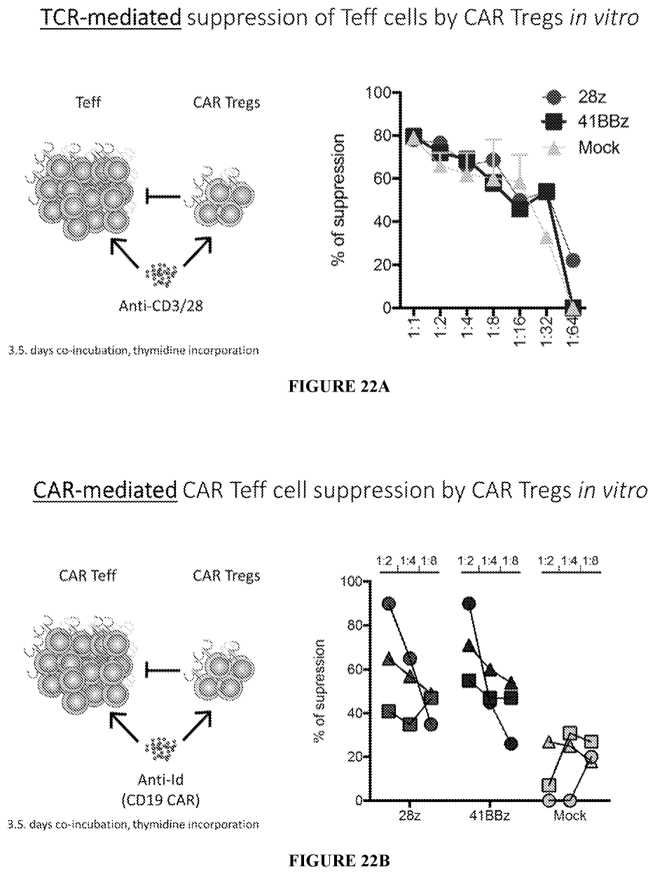

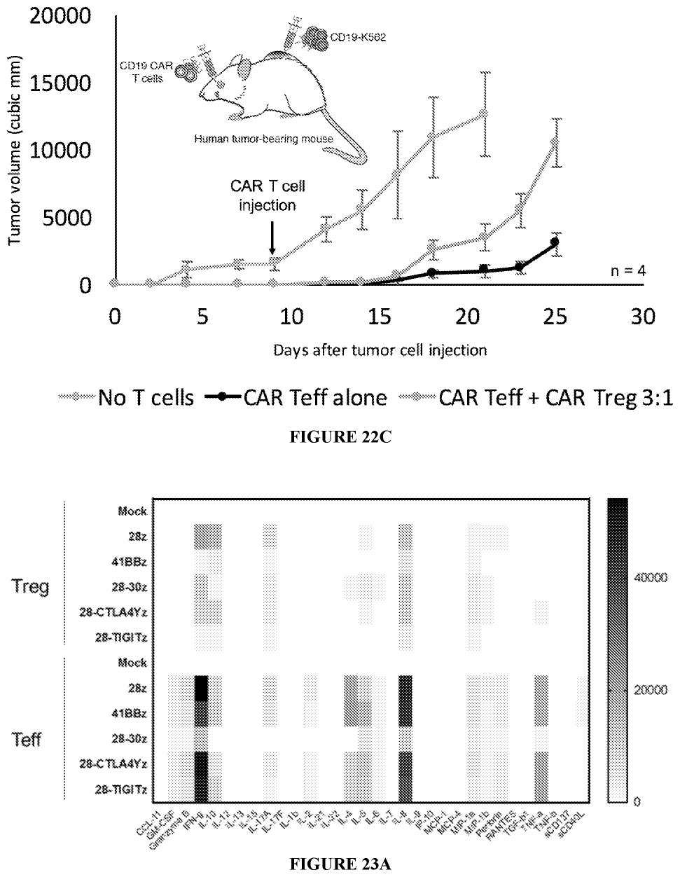

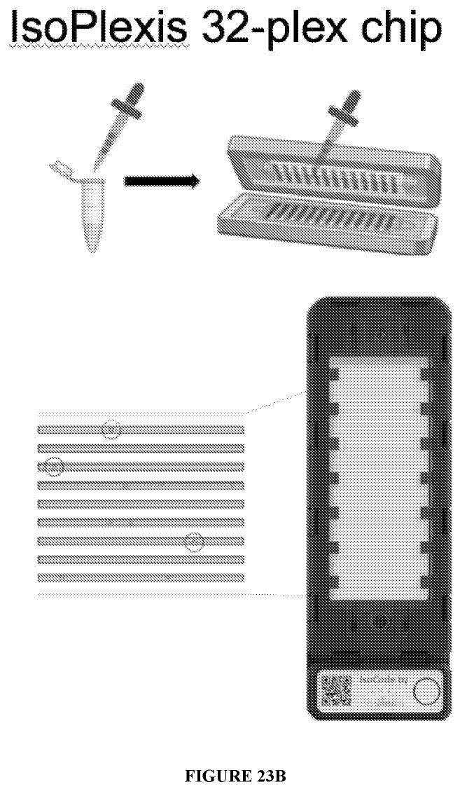

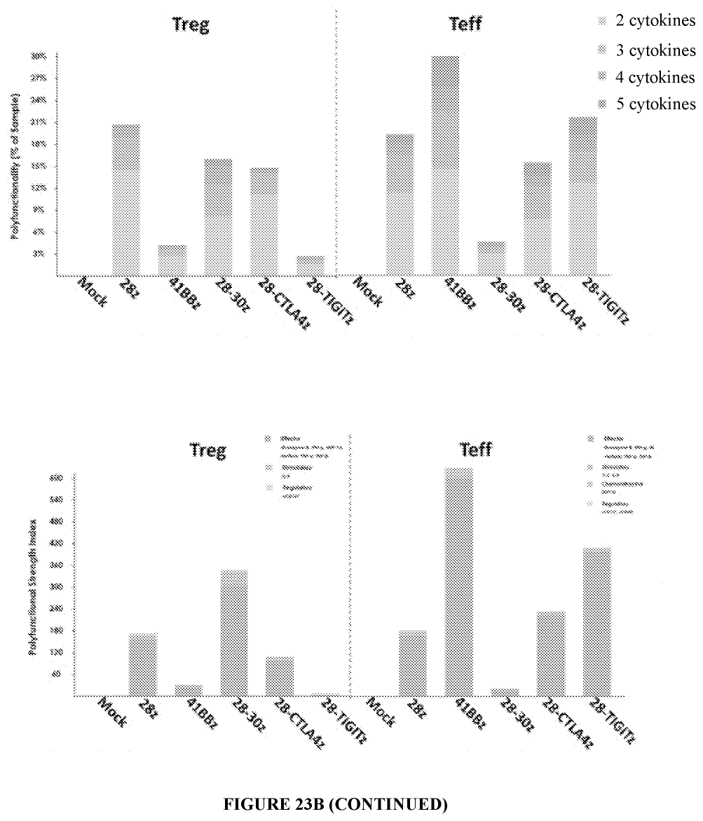

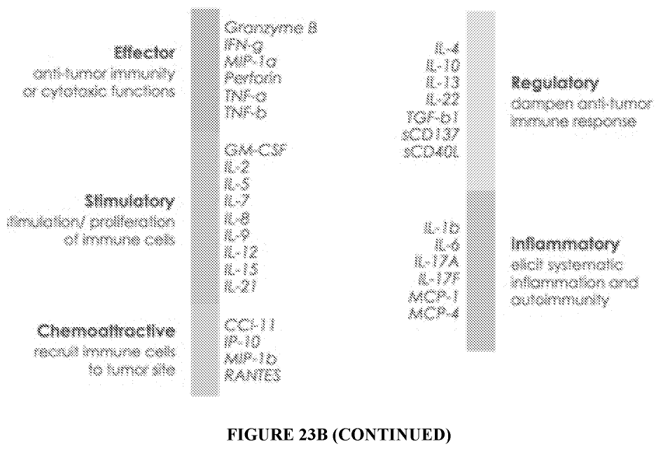

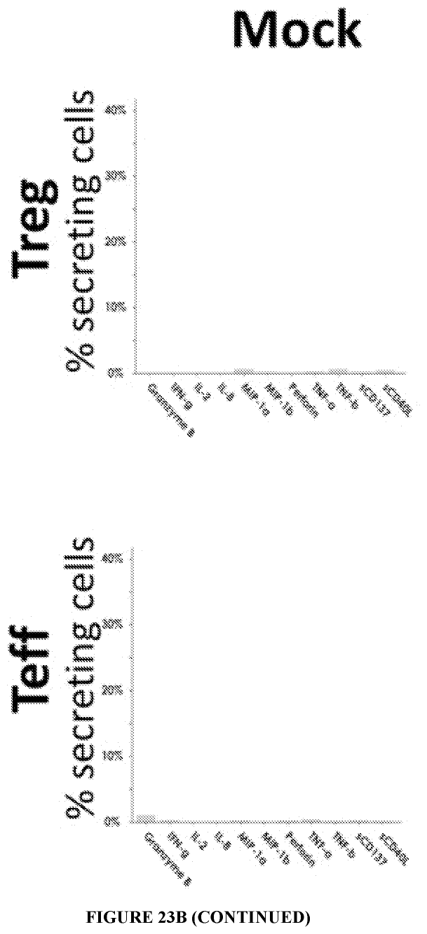

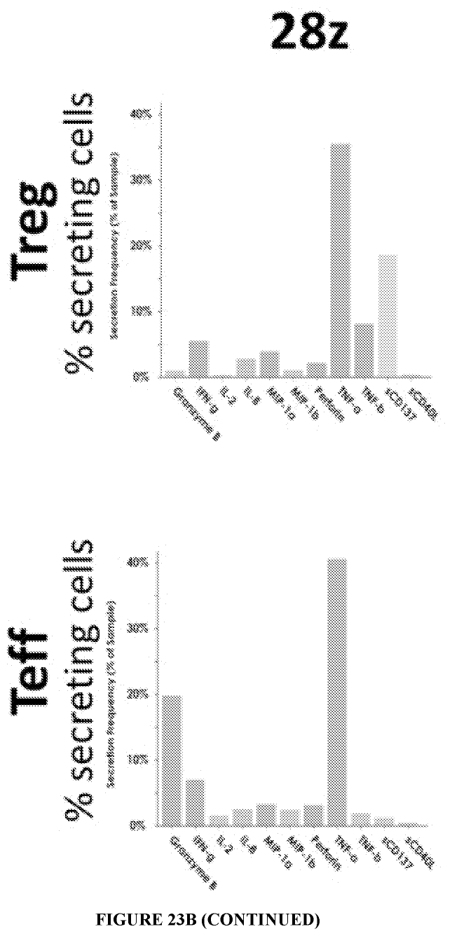

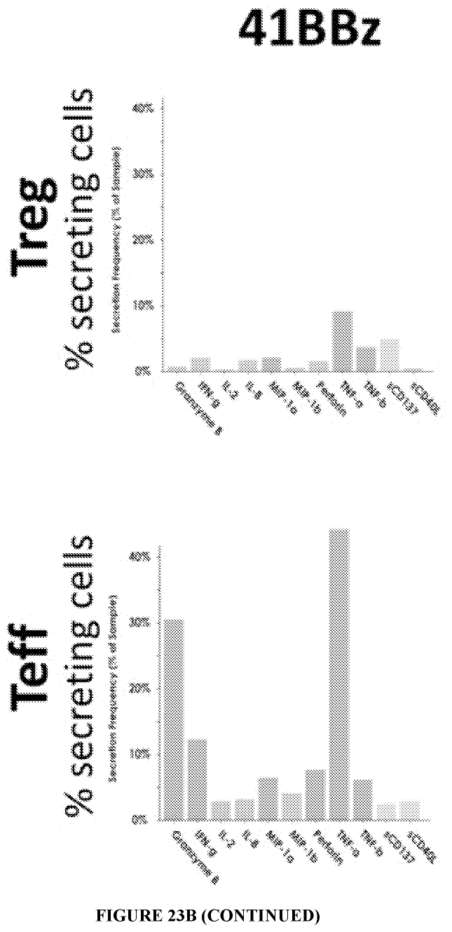

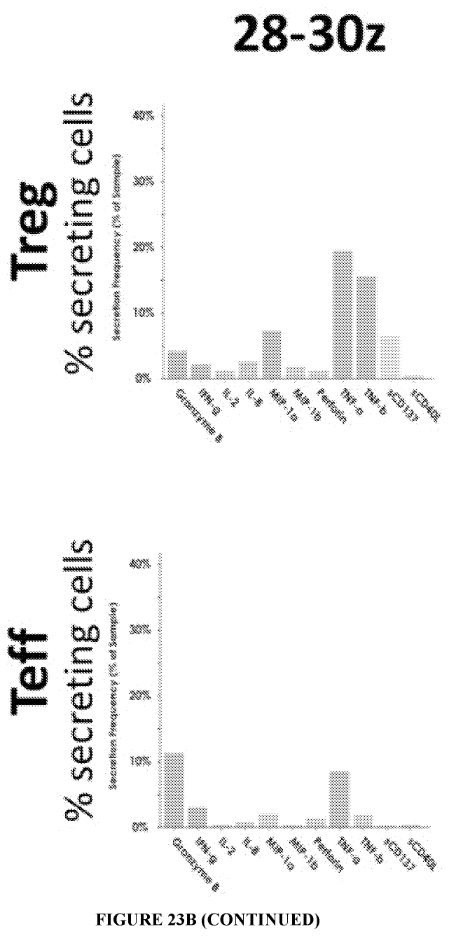

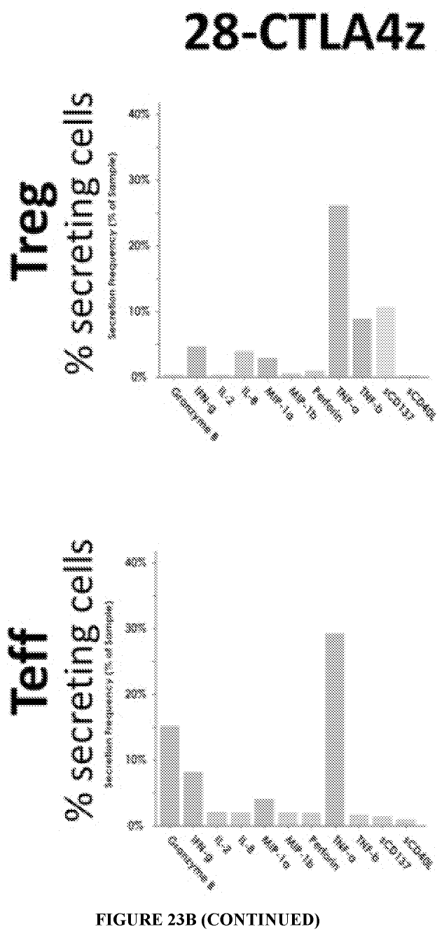

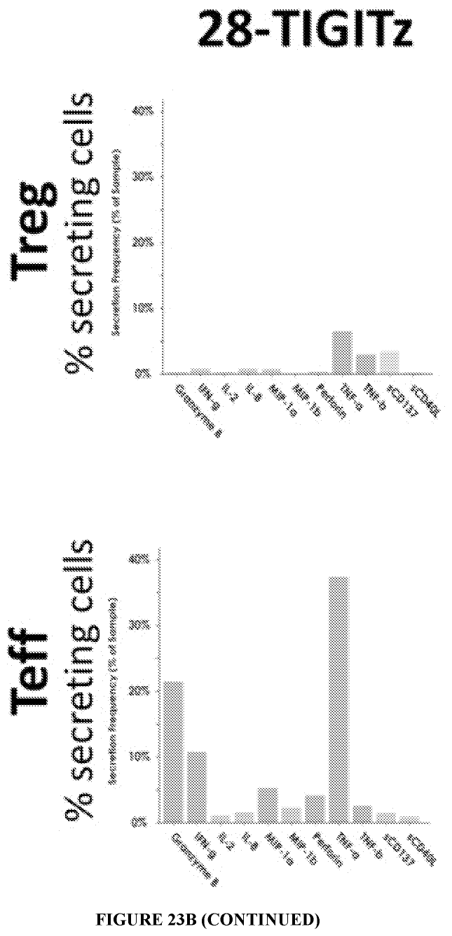

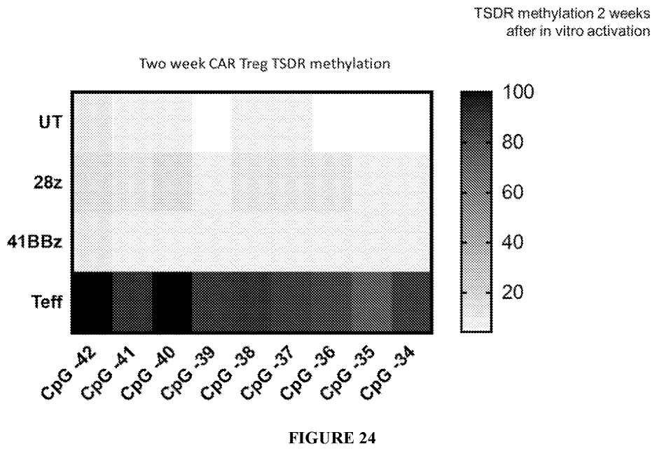

Next, the suppressive capacity of CAR-expressing Tregs in vivo was tested. The immunodeficient NSG mouse model was utilized to measure the efficacy of human CAR-expressing Tregs transduced with the various CARs described above. 8-12 week old NSG mice were injected subcutaneously with luciferase-labeled CD19-K562 cells. Ten days later, total CD19 CAR-expressing T cells were injected intravenously (retro-orbital route) either with or without CD19 CAR-expressing Tregs. CAR-expressing T cells led to tumor rejection (as assessed by luciferase activity and tumor volume) within 2 weeks; different CAR-expressing Tregs should prevent the rejection to varying degrees, as demonstrated in pilot experiments graphed in FIGS. 22A, 22B, 22C.

Planned experiments include: Quantifying CAR-expressing Teff (and CAR-expressing Treg) cell expansion with CARs with different CD3 chains and CD28 mutants (and others); dissecting signaling domains of 41BB, CD30, TIGIT, CTLA4, amongst others, to use shortened/modified versions of these domains in CARs for Tregs; design additional growth factor receptor CAR signaling domains, from, but not limited to, IL2Rb (CD122), generally important for Tregs, and IL-33 receptor (ST2), important for tissue-resident Tregs; In vivo suppression with all the different CAR Tregs described herein; In vitro suppression of effector T cell proliferation and killing by all the different CAR Tregs described herein; Cytokine production (e.g. IL-10, IFNγ) by all CAR Tregs described herein and CAR Teff cells described herein; CAR Treg cell fate lineage stability (TSDR analysis); Test these signaling architectures with a different CAR or other chimeric receptor specificity, including allo HLA antigen; Test efficacy of CAR-expressing Tregs in humanized mouse models of type 1 diabetes and/or other autoimmune diseases; Test efficacy of CAR-expressing Tregs in mouse models of transplantation.

### REFERENCES

1. B. A. Irving, A. Weiss, The cytoplasmic domain of the T cell receptor zeta chain is sufficient to couple to receptor-associated signal transduction pathways. Cell 64, 891-901 (1991).
2. T. Brocker, K. Karjalainen, Signals through T cell receptor-zeta chain alone are insufficient to prime resting T lymphocytes. J Exp Med 181, 1653-59 (1995).
3. M. C. Milone et al., Chimeric receptors containing CD137 signal transduction domains mediate enhanced survival of T cells and increased antileukemic efficacy in vivo. Mol Ther 17, 1453-64 (2009).
4. C. M. Kowolik et al., CD28 costimulation provided through a CD19-specific chimeric antigen receptor enhances in vivo persistence and antitumor efficacy of adoptively transferred T cells. Cancer Res 66, 10995-11004 (2006).
5. A. H. Long et al., 4-1BB costimulation ameliorates T cell exhaustion induced by tonic signaling of chimeric antigen receptors. Nat Med 21, 581-90 (2015).
6. A. A. Hombach, J. Heiders, M. Foppe, M. Chmielewski, H. Abken, OX40 costimulation by a chimeric antigen receptor abrogates CD28 and IL-2 induced IL-10 secretion by redirected CD4(+) T cells. Oncoimmunology 1:458-66 (2012).
7. D. Yan, J. Farache, M. Mingueneau, D. Mathis, C. Benoist, Imbalanced signal transduction in regulatory T cells expressing the transcription factor FoxP3. Proc Natl Acad Sci USA 112, 14942-47 (2015).
8. M. A. Gavin, S. R. Clarke, E. Negrou, A. Gallegos, A. Rudensky, Homeostasis and anergy of CD4(+)CD25(+) suppressor T cells in vivo. Nat Immunol 3, 33-41 (2002).
9. J. Holst et al., Scalable signaling mediated by T cell antigen receptor-CD3 ITAMs ensures effective negative selection and prevents autoimmunity. Nat Immunol 9, 658-666 (2008).
10. R. Zhang, C. M. Borges, M. Y. Fan, J. E. Harris, L. A. Turka, Requirement for CD28 in Effector Regulatory T Cell Differentiation, CCR6 Induction, and Skin Homing. J Immunol 195, 4154-4161 (2015).
11. B. Salomon et al., B7/CD28 costimulation is essential for the homeostasis of the CD4+CD25+ immunoregulatory T cells that control autoimmune diabetes. Immunity 12, 431-440 (2000).
12. Q. Tang et al., Cutting edge: CD28 controls peripheral homeostasis of CD4+CD25+ regulatory T cells. J Immunol 171, 3348-3352 (2003).
13. T. M. Kundig et al., Duration of TCR stimulation determines costimulatory requirement of T cells. Immunity 5, 41-52 (1996).
14. J. S. Boomer, J. M. Green, An enigmatic tail of CD28 signaling. Cold Spring Harb Perspect Biol 2, a002436 (2010).
15. L. F. Dodson et al., Targeted knock-in mice expressing mutations of CD28 reveal an essential pathway for costimulation. Mol Cell Biol 29, 3710-3721 (2009).
16. J. S. Boomer, C. M. Deppong, D. D. Shah, T. L. Bricker, J. M. Green, Cutting edge: A double-mutant knockin of the CD28 YMNM and PYAP motifs reveals a critical role for the YMNM motif in regulation of T cell proliferation and Bcl-xL expression. J Immunol 192, 3465-3469 (2014).
17. L. E. Marengere et al., The SH3 domain of Itk/Emt binds to proline-rich sequences in the cytoplasmic domain of the T cell costimulatory receptor CD28. J Immunol 159, 3220-3229 (1997).
18. S. Ogawa et al., CD28 signaling in primary CD4(+) T cells: identification of both tyrosine phosphorylation-dependent and phosphorylation-independent pathways. Int Immunol 25, 671-681 (2013).
19. S. J. Bensinger et al., Distinct IL-2 receptor signaling pattern in CD4+CD25+ regulatory T cells. J Immunol 172, 5287-5296 (2004).
20. M. Battaglia, A. Stabilini, M. G. Roncarolo, Rapamycin selectively expands CD4+CD25+FoxP3+ regulatory T cells. Blood 105, 4743-4748 (2005).
21. D. Valmori et al., Rapamycin-mediated enrichment of T cells with regulatory activity in stimulated CD4+ T cell cultures is not due to the selective expansion of naturally occurring regulatory T cells but to the induction of regulatory functions in conventional CD4+ T cells. J Immunol 177, 944-949 (2006).
22. A. C. Anderson, N. Joller, V. K. Kuchroo, Lag-3, Tim-3, and TIGIT: Co-inhibitory Receptors with Specialized Functions in Immune Regulation. Immunity 44, 989-1004 (2016).
23. L. S. Walker, D. M. Sansom, Confusing signals: recent progress in CTLA-4 biology. Trends Immunol 36, 63-70 (2015).
24. A. Vasanthakumar et al., The TNF Receptor Superfamily-NF-kappaB Axis Is Critical to Maintain Effector Regulatory T Cells in Lymphoid and Non-lymphoid Tissues. Cell Rep 20, 2906-2920 (2017).
25. Y. Grinberg-Bleyer et al., Pathogenic T cells have a paradoxical protective effect in murine autoimmune diabetes by boosting Tregs. J Clin Invest 120, 4558-4568 (2010).
26. M. D. Rosenblum, S. S. Way, A. K. Abbas, Regulatory T cell memory. Nat Rev Immunol 16, 90-101 (2016).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A chimeric antigen receptor (CAR) comprising an antigen specific binding domain, a spacer domain, a transmembrane domain, and an intracellular signaling region, the signaling region comprising a primary signaling domain, optionally derived from a CD3 chain domain, and a second signaling domain which is a costimulatory or inhibitory signaling domain of a protein selected from the group consisting of: CD28, ICOS, CTLA4, 41BB, CD27, CD30, CD132, OX-40, TACI, GITR, HVEM, TIM3, TIGIT, other TNFR superfamily members, and derivatives, mutants, variants, fragments and combinations thereof.

2. The chimeric antigen receptor of claim 1, wherein the antigen specific binding domain comprises an antibody, a T cell receptor variable region, soluble T cell receptor, aptamer, nanobody, receptors, fragments or combinations thereof.

3. The chimeric antigen receptor of claim 2, wherein the T cell receptor or antibody is a single chain fragment.

4. The chimeric antigen receptor of claim 2, wherein the single chain fragment is a single chain variable fragment (scFv).

5. The chimeric antigen receptor of claim 1, wherein the primary signaling domain is or comprises the CD3 chain domain, wherein the CD3 chain is selected from the group consisting of: a CD3 zeta (CD3ζ) chain, a CD3 gamma (CD3γ) chain, a CD3 delta (CD3δ) chain, a CD3 epsilon (CD3ε) chain, derivatives, mutants, variants, fragments and combinations thereof.

6. The chimeric antigen receptor of claim 1, wherein the signaling domain optionally comprises an Fcγ domain, derivatives, mutants, variants, fragments and combinations thereof.

7. The chimeric antigen receptor of claim 1, wherein the second signaling domain is a costimulatory domain derived from a protein selected from the group consisting of: CD28, ICOS, CTLA4, 41BB, CD27, CD30, derivatives, mutants, variants, fragments and combinations thereof.

8. (canceled)

9. (canceled)

10. (canceled)

11. The chimeric antigen receptor of claim 1, wherein the signaling domain is selected from the group consisting of: (i) CD28, ICOS, CTLA4, 41BB or combinations thereof; (ii) at least one domain selected from TACI, HVEM, GITR, OX40, CD27, CD30; and (iii) a CD3 zeta (CD3ζ) chain, a CD3 gamma (CD3γ) chain, a CD3 delta (CD3δ) chain, a CD3 epsilon (CD3ε) chain, or combinations thereof.

12. The chimeric antigen receptor of claim 1, wherein the signaling domain is selected from the group consisting of: (i) CD28, 41BB or a combination thereof, (ii) at least one domain selected from CTLA4, PD1, TIM3, LAG3, or TIGIT; and (iii) a CD3 zeta (CD3ζ) chain, a CD3 gamma (CD3γ) chain, a CD3 delta (CD3δ) chain, a CD3 epsilon (CD3ε) chain, Fcγ or combinations thereof.

13. An isolated T cell that is modified to express: a chimeric antigen receptor (CAR) comprising an antigen specific binding domain, a spacer domain, a transmembrane domain, and an intracellular signaling region, the signaling region comprising a primary signaling domain, optionally derived from a CD3 chain domain, wherein the CD3 chain is selected from the group consisting of: a CD3 zeta (CD3ζ) chain, a CD3 gamma (CD3γ) chain, a CD3 delta (CD3δ) chain, a CD3 epsilon (CD3ε) chain, derivatives, mutants, variants, fragments and combinations thereof, and a second signaling domain which is a costimulatory or inhibitory signaling domain of a protein selected from the group consisting of: CD28, ICOS, CTLA4, 41BB, CD27, CD30, CD132, OX-40, TACI, GITR, HVEM, TIM3, TIGIT, other TNFR superfamily members, and derivatives, mutants, variants, fragments and combinations thereof.

14. The isolated T cell of claim 13, wherein the antigen specific binding domain comprises an antibody, a T cell receptor variable region, soluble T cell receptor, aptamer, nanobody, receptors, fragments or combinations thereof.

15. The isolated T cell of claim 14, wherein the antibody is a single chain variable fragment (scFv).

16. (canceled)

17. (canceled)

18. The isolated T cell of claim 13, wherein the signaling domain optionally comprises an Fcγ domain, derivatives, mutants, variants, fragments and combinations thereof.

19. (canceled)

20. The isolated T cell of claim 13, wherein the second signaling domain is a costimulatory domain derived from a protein selected from the group consisting of: CD28, ICOS, CTLA4, 41BB, CD27, CD30, derivatives, mutants, variants, fragments and combinations thereof.

21. The isolated T cell of claim 13, wherein the second signaling domain is an inhibitory signaling domain of a protein selected from the group consisting of: CTLA4, PD-1, TIM3, LAG3, TIGIT, derivatives, mutants, variants, fragments and combinations thereof.

22. (canceled)

23. (canceled)

24. (canceled)

25. The isolated T cell of claim 13, wherein the signaling domain is selected from the group consisting of: (i) CD28, ICOS, CTLA4, 41BB or combinations thereof; (ii) at least one domain selected from TACT, HVEM, GITR, OX40, CD27, CD30; and (iii) a CD3 zeta (CD3ζ) chain, a CD3 gamma (CD3γ) chain, a CD3 delta (CD3δ) chain, a CD3 epsilon (CD3ε) chain, Fcγ domain or combinations thereof.

26. The isolated T cell of claim 13, wherein the signaling domain is selected from the group consisting of: (i) CD28, 41BB or a combination thereof, (ii) at least one domain selected from CTLA4, PD1, TIM3, LAG3, or TIGIT (iii) a CD3 zeta (CD3ζ) chain, a CD3 gamma (CD3γ) chain, a CD3 delta (CD3δ) chain, a CD3 epsilon (CD3ε) chain, Fcγ domain or combinations thereof.

27. The isolated T cell of claim 13, wherein the Treg cell is CD4⁺CD25⁺ CD127⁻, FOXP3⁺ and Helios⁺.

28. A chimeric antigen receptor comprising an antigen specific binding domain and at least one signaling region, wherein the signaling region consists of (i) CD28; (ii) 41BB, TACI, HVEM, GITR, OX40, CD27 or CD30; and (iii) a CD3ζ chain; or,
a chimeric antigen receptor comprising an antigen specific binding domain and at least one signaling region, wherein the signaling region consists of (i) CD28; (ii) CTLA4, PD-1, TIM3, LAG3 or TIGIT; and (iii) a CD3ζ chain; or,
a chimeric antigen receptor comprising an antigen specific binding domain and at least one signaling region, wherein the signaling region consists of (i) CD28; (ii) CD132; and (iii) a CD3ζ chain; or,
a chimeric antigen receptor comprising an antigen binding domain and at least one signaling region, wherein the signaling region consists of (i) ICOS; (ii) 41BB, and (iii) a CD3ζ chain; or,
a chimeric antigen receptor comprising an antigen specific binding domain and at least one signaling region, wherein the signaling region consists of (i) CTLA4; (ii) 41BB, and (iii) a CD3ζ chain.

29. (canceled)

30. (canceled)

31. (canceled)

32. (canceled)

33. (canceled)

34. (canceled)

35. A method of treating a subject suffering from an autoimmune or inflammatory disease or disorder, comprising:
isolating and separating CD4⁺ T regulatory cells (Tregs) from a biological sample, wherein a biological sample is obtained from one or more sources comprising: autologous, allogeneic, haplotype matched, haplotype mismatched, haplo-identical, or xenogeneic cell lines, or combinations thereof and, wherein the Treg cells are CD4⁺CD25⁺CD127⁻;
contacting the Treg cells with an expression vector encoding a chimeric antigen receptor (CAR) which specifically binds to an antigen associated with an autoimmune response and/or suppresses an effector T cell (Teff) or inflammatory immune response;
stimulating the Treg with a specific antigen to obtain a therapeutically effective number of antigen-specific Treg cells; and,
reinfusing the Treg into the subject suffering from an autoimmune or inflammatory disease or disorder; or,
providing an isolated T cell wherein expression of pro-inflammatory cytokines is suppressed, wherein said isolated T cell is modified to express: a chimeric antigen receptor (CAR) comprising an antigen specific binding domain, a spacer domain, a transmembrane domain, and an intracellular signaling region, the signaling region comprising a primary signaling domain, optionally derived from a CD3 chain domain, and a second signaling domain of a protein selected from the group consisting of 41BB and TIGIT;
infusing the isolated T cell into a subject suffering from an autoimmune or inflammatory disease or disorder,
thereby treating the subject.

36. A method of treating a subject suffering from graft versus host disease, or is undergoing an organ transplantation, comprising:
isolating and separating CD4⁺ T regulatory cells (Tregs) from a biological sample, wherein a biological sample is obtained from one or more sources comprising: autologous, allogeneic, haplotype matched, haplotype mismatched, haplo-identical, or xenogeneic cell lines, or combinations thereof and, wherein the Treg cells are CD4⁺CD25⁺CD127⁻;
contacting the Treg cells with an expression vector encoding a chimeric antigen receptor (CAR) which suppresses an effector T cell (Teff) immune response;
stimulating the Treg with a specific antigen to obtain a therapeutically effective number of antigen-specific Treg cells; and,
reinfusing the Treg into the subject,
thereby treating the subject.

37. (canceled)

38. (canceled)

39. (canceled)

40. (canceled)

41. (canceled)

42. (canceled)
